(19) United States
(12) Patent Application Publication
(10) Pub. No.: US 2015/0328232 A1
(43) Pub. Date: Nov. 19, 2015

(54) PROSTACYLIN COMPOSITIONS AND METHODS FOR USING THE SAME

(71) Applicant: INSMED INCORPRATION, Bridgewater, NJ (US)

(72) Inventors: Vladimir Malinin, Plainsboro, NJ (US); Walter Perkins, Pennington, NJ (US); Franziska Leifer, Princeton, NJ (US); Donna M. Omiatek, Belle Mead, NJ (US); Jane Ong, Franklin Park, NJ (US); Renu Gupta, Moorestown, NJ (US); Zhili Li, Kendall Park, NJ (US)

(21) Appl. No.: 14/648,632

(22) PCT Filed: Dec. 2, 2013
(86) PCT No.: PCT/US13/72647

§ 371 Date: May 29, 2015

Related U.S. Application Data
(60) Provisional application No. 61/732,223, filed on Nov. 30, 2012.

(51) Int. Cl. A61K 31/5575; A61K 47/14; A61K 47/28; A61K 9/51; A61K 47/18

(52) U.S. Cl. CPC A61K 31/5575; A61K 9/5123; A61K 47/186; A61K 47/28; A61K 47/14

(57) ABSTRACT

The present invention relates to pharmaceutical compositions comprising a prostacyclin, a cationic compound, and a surfactant. Particulate compositions, including liposomal, solid nanoparticulate prostacyclin compositions, including treprostinil formulations comprising cationic compound and the surfactant are also described. The present invention also relates to a system comprising the pharmaceutical composition and an inhalation device. Methods for treating pulmonary hypertension and portopulmonary hypertension with the compositions and systems described herein are also provided.

### CROSS REFERENCE TO RELATED APPLICATION

This application claims priority from U.S. Provisional Application Ser. No. 61/732,223, filed Nov. 30, 2012, which is hereby incorporated by reference in its entirety for all purposes.

### BACKGROUND OF THE INVENTION

Pulmonary hypertension (PH) is characterized by an abnormally high blood pressure in the lung vasculature. It is a progressive, lethal disease that leads to heart failure and can occur in the pulmonary artery, pulmonary vein, or pulmonary capillaries. Symptomatically patients experience shortness of breath, dizziness, fainting, and other symptoms, all of which are made worse by exertion. There are multiple causes, and can be of unknown origin, idiopathic, and can lead to hypertension in other systems, for example, portopulmonary hypertension in which patients have both portal and pulmonary hypertension.

Pulmonary hypertension has been classified into five groups by the World Health Organization (WHO). Group I is called pulmonary arterial hypertension (PAH), and includes PAH that has no known cause (idiopathic), inherited PAH (i.e., familial PAH or FPAH), PAH that is caused by drugs or toxins, and PAH caused by conditions such as connective tissue diseases, HIV infection, liver disease, and congenital heart disease. Group II pulmonary hypertension is characterized as pulmonary hypertension associated with left heart disease. Group III pulmonary hypertension is characterized as PH associated with lung diseases, such as chronic obstructive pulmonary disease and interstitial lung diseases, as well as PH associated with sleep-related breathing disorders (e.g., sleep apnea). Group IV PH is PH due to chronic thrombotic and/or embolic disease, e.g., PH caused by blood clots in the lungs or blood clotting disorders. Group V includes PH caused by other disorders or conditions, e.g., blood disorders (e.g., polycythemia vera, essential thrombocythemia), systemic disorders (e.g., sarcoidosis, vasculitis), metabolic disorders (e.g., thyroid disease, glycogen storage disease).

Group I PH, or pulmonary arterial hypertension (PAH) is a dyspnea-fatigue syndrome defined by an isolated increase in pulmonary vascular resistance (PVR), which leads to progressive right heart failure. PAH occurs in association with a variety of conditions which include connective tissue diseases (CTD), congenital heart diseases (CHD), portal hypertension, human immunodeficiency viral (HIV) infection, and intake of appetite suppressant drugs, mainly fenfluramines.

PAH afflicts 30,000-40,000 people in U.S. with 20,000-25,000 under treatment. It is a progressive disease ultimately causing patients to die of heart failure. Despite available treatments, the one-year mortality rate is 15%. The current treatment for PAH is progressive combination therapy usually starting with calcium channel blockers (CCB), followed by phosphodiesterase-5 (PDE-5) inhibitors. In some instances, endothelin receptor antagonists (ERA) and prostanoids (e.g., prostacyclins) are added as the disease progresses. Prostanoids are perceived to be the most effective class of drugs for PAH, but their effectiveness is limited due to significant toxicity/tolerability issues and inconvenient dosing regimens (e.g., daily IV infusions or 4-9 inhalations per day). The current inhaled prostanoid products are iloprost (Ventavis®, 6-9 inhalation treatments per day) and treprostinil (Tyvaso®, 4 inhalation treatments per day, spaced 4 hours apart). While longer than that for iloprost, the half-life of treprostinil is still relatively short necessitating dosing every 4 hours over the time patients are awake. For the Tyvaso® (treprostinil) patient, dosing compliance is a major issue.

Portopulmonary hypertension is defined by the coexistence of portal and pulmonary hypertension, and is a serious complication of liver disease. The diagnosis of portopulmonary hypertension is based on hemodynamic criteria: (1) portal hypertension and/or liver disease (clinical diagnosis-ascites/varices/splenomegaly), (2) mean pulmonary artery pressure >25 mmHg at rest, (3) pulmonary vascular resistance >240 dynes s cm⁻⁵, (4) pulmonary artery occlusion pressure <15 mmHg or transpulmonary gradient >12 mmHg.

Treprostinil is a tricyclic benzindene analogue of prostacyclin which has a similar antiplatelet aggregation and vasodilatory actions including acute pulmonary vasodilatation. Treprostinil is rapidly and completely absorbed after subcutaneous administration with an absolute bioavailability of 100%, and has an elimination half-life of 4.6 hours. Continuous subcutaneous infusion of treprostinil is associated with steady state plasma concentrations after about 10 hours with administration rates of 1.25 to 22 ng/kg/min. Approximately 79% of the administered drug is excreted in urine either as unchanged drug (4%) or an identifiable metabolite (64%). The clearance of treprostinil is decreased up to 80% in patients with hepatic insufficiency and therefore requires cautious dosing in patients with PAH associated with liver disease.

The dose of intravenous treprostinil has been reported to be at least double that of subcutaneous infusion to maintain the same efficacy. In addition, intravenous treprostinil appears to expose PAH patients to series of complications including blood stream infections, thrombosis, and delivery systems malfunctions resulting in poorly tolerated rapid overdosing or under dosing.

Epoprostenol is another prostacyclin that has also been used for the treatment of PAH patients. However, current treatments are not ideal. For example, epoprostenol must be administered as a continuous infusion because of its instability and very short half-life (2-7 min).

The complicated delivery system and potential side effects associated with prostacyclins have deterred some patients and caregivers from utilizing this class of agents. Therefore, more stable prostacyclin compositions and routes of administrations are needed to provide a more efficient prostacyclin therapy. The present invention addresses this and other needs.

### SUMMARY OF THE INVENTION

The present invention relates generally to pharmaceutical compositions comprising a prostacyclin or analog thereof, systems comprising the same, as well as methods for using the pharmaceutical compositions and systems for the treatment of various indications, for example pulmonary hypertension (e.g., pulmonary arterial hypertension, chronic thromboembolic pulmonary hypertension) and portopulmonary hypertension.

A first aspect of the invention relates to a pharmaceutical composition comprising a prostacyclin. In one embodiment, the pharmaceutical composition comprises a prostacyclin (e.g., treprostinil) or analog thereof, a cationic compound and a surfactant. In another embodiment, the pharmaceutical composition comprises a prostacyclin (e.g., treprostinil) or analog thereof, a cationic compound, a surfactant (e.g., a PEGylated lipid) and a hydrophobic additive (e.g., squalane). In one embodiment, the cationic compound is a cationic lipid, cationic polymer, or an inorganic ion. In a further embodiment, the prostacyclin is treprostinil. In even a further embodiment, the inorganic ion is an aluminum ion. In yet a further embodiment, the hydrophobic additive is squalane.

In one embodiment, the pharmaceutical composition comprises a plurality of particles comprising the prostacyclin or analog thereof and the cationic compound. The mean diameter of the plurality of particles, in one embodiment, is about 500 nm or less, about 400 nm or less, about 300 nm or less, about 200 nm or less, about 150 nm or less, about 100 nm or less, or about 50 nm or less. In another embodiment, the mean diameter of the plurality of particles is about 100 nm to about 500 nm or less, about 200 nm to about 400 nm, about 50 nm to about 300 nm, about 50 nm to about 500 nm or about 100 nm to about 400 nm. In a further embodiment, the surfactant is associated with one or more of the plurality of particles. In a further embodiment, the plurality of particles is a plurality of solid particles. In another embodiment, the plurality of particles comprise solid colloidal particles, polymer-lipid hybrid nanoparticles, nanostructured lipid carriers, polymeric microspheres, nanoparticles, micelles, liposomes, solid lipid particles, solid lipid nanoparticles, or a combination thereof. In a further embodiment, the plurality of particles comprises solid lipid nanoparticles.

In one embodiment, the surfactant is associated with one or more of the plurality of particles in the pharmaceutical composition. The surfactant, in a further embodiment, is a PEGylated lipid.

The prostacyclin or analog thereof, in one embodiment, is treprostinil, epoprostenol, or iloprost.

In yet another embodiment, the cationic compound is multicationic. The multicationic compound, in one embodiment, is an ion or a lipid. In one embodiment, the multicationic compound is selected from alkyl-ammonium, alkyl-polyammonium, linear polyamine, linear polyethylenimine, branched polyethylcnimine, poly-L-lysine, trimethyl-poly-glucosamine, or a multivalent metal ion. In a further embodiment, the cationic compound is dioctadecyldimethyl ammonium bromide (diC18dMA), dimethyldihexadecylammonium chloride, N-[1-(2,3-dioleoyloxy)propyl]-N,N,N-trimethylammonium methyl sulfate (DOTAP), N-[1-(2,3-dioleyloxyl)propyl]-N,N,N-trimethylammonium chloride (DOTMA), 1,2-distearoyl-3-(trimethylammonio)propane chloride (DSTAP), dimyristoyltrimethylammonium propane (DMTAP). The at least one cationic compound in other embodiments, is N,N′-dihexadecyl-1,2-ethanediamine, tetraethylhexadecane-1,16-diamine, or hexadecane-1,16-bis(trimethylammonium bromide). In one embodiment, the cationic compound is a one metal ion, for example, aluminum, magnesium, beryllium, strontium, barium, or calcium. In one embodiment, the cationic compound is a cationic lipid (e.g., diC18dMA).

As provided above, in one aspect, the present invention relates to a pharmaceutical composition comprising a prostacyclin or analog thereof, a cationic compound, and a surfactant. The at least one surfactant, in one embodiment, is nonionic. The surfactant, in one embodiment, is polyoxyethyleneglycol-lipid (also referred to as a “PEGylated lipid” or “PEG-lipid”), polyoxypropyleneglycol-lipid, glucoside-lipid, glycerol-lipid, or polysorbate-lipid. In one embodiment, the pharmaceutical composition comprises a plurality of particles comprising the prostacyclin or analog thereof, the at least one cationic compound and the at least one surfactant. In a further embodiment, the plurality of particles comprises solid lipid nanoparticles.

In one aspect of the invention, an effective amount of the prostacyclin composition described herein is administered to a patient in need thereof, for example for the treatment of pulmonary hypertension or portopulmonary hypertension. In one embodiment, the administration is intranasal, oral, parenteral, by injection (e.g., subcutaneous, intravenous, intramuscular), by inhalation, or by infusion. In another embodiment the prostacyclin composition is delivered in the lungs of the patient via inhalation. In one embodiment, the pharmaceutical composition is administered in a once-a-day dosing or a twice-a-day dosing regimen for the treatment of pulmonary hypertension (e.g., pulmonary arterial hypertension, chronic thromboembolic pulmonary hypertension) or portopulmonary hypertension.

In yet another embodiment, the pharmaceutical composition is administered to the lungs of a patient via an inhalation device, e.g., a nebulizer. In a further embodiment, upon aerosolization of the composition (e.g., with a nebulizer or other aerosol generator), the aerosolized composition has an average aerosol droplet size, i.e., a mass median aerodynamic diameter (MMAD) of less than 10 μm, as measured by cascade impaction. In a further embodiment, upon aerosolization, the aerosol has a MMAD of less than about 8 μm, less than about 7 μm, less than about 6 μm, less than about 5 μm, less than about 4 μm, less than about 3 μm or less than about 2 μm, as measured by cascade impaction.

Another aspect of the present invention relates to a system for treating or providing prophylaxis against pulmonary hypertension, for example, pulmonary arterial hypertension. The system comprises, in one embodiment, a pharmaceutical composition comprising a prostacyclin or analog thereof, a cationic compound, a surfactant; and an inhalation device (e.g., a dry powder inhaler or a nebulizer). The inhalation device in one embodiment, is an electronic nebulizer that is portable and easy to use. In one embodiment, the nebulizer is disposable. In a further embodiment, the pharmaceutical composition comprises a hydrophobic additive. In one embodiment, the pharmaceutical composition comprises a plurality of particles, e.g., solid lipid nanoparticles comprising the prostacyclin or analog thereof, the cationic compound and the surfactant. In one embodiment, the inhalation device is a nebulizer. In one embodiment, the prostacyclin is treprostinil. In a further embodiment, the cationic compound is a metal ion, a polymer, or a lipid. In one embodiment, the inhalation device (e.g., nebulizer) generates an aerosol of the pharmaceutical composition at a rate of about 0.1 to 1.0 mL/min. In one embodiment, the mass median aerodynamic diameter of the aerosol droplets is about 1 μm to 5 jm as measured by cascade impaction. In one embodiment, the fine particle fraction (FPF) of the aerosol is greater than or equal to about 50%, or about 60% or about 65%, as measured by cascade impaction, for example by the Anderson Cascade Impactor (ACI) or the Next Generation Impactor (NGI).

Another aspect of the present invention relates to a method for treating or providing prophylaxis against pulmonary hypertension in a patient in need thereof. In one embodiment, the patient is administered a prostacyclin composition described herein intravenously, subcutaneously or via inhalation. In one embodiment, the method involves aerosolizing the pharmaceutical composition and delivering the aerosol to the lungs of the patient in need thereof. In one embodiment, the pulmonary hypertension is group I pulmonary hypertension (i.e., PAH). In another embodiment, the pulmonary hypertension is group II, group III, group IV or group V pulmonary hypertension. The method, in one embodiment, involves administering an effective amount of the pharmaceutical composition described herein to a patient in need of treatment for pulmonary hypertension.

Another aspect of the present invention relates to a method for treating or providing prophylaxis against portopulmonary hypertension in a patient in need thereof. The method, in one embodiment, comprises administering an effective amount of one of the prostacyclin compositions described herein to the patient in need of treatment for portopulmonary hypertension. In one embodiment, administration is via inhalation, subcutaneous or intravenous.

In one embodiment, the pharmaceutical composition is administered once-a-day or twice-a-day to the patient in need thereof. In embodiments where the composition is administered via inhalation, upon administration, in one embodiment, the prostacyclin (e.g., treprostinil) or analog thereof is released in the lungs over a time period ranging from about 6 hours to about 48 hours, for example about 12 hours to about 36 hours or about 12 hours to about 24 hours.

Another aspect of the present invention relates to an aerosol comprising a plurality of solid particles of one or more of the pharmaceutical compositions described herein. In one embodiment, the plurality of solid particles has an average diameter of less than 200 nm as measured by light scattering. In another embodiment, the plurality of solid particles has an average diameter of about 1 nm to about 1000 nm, or about 10 nm to about 500 nm, or about 100 nm to about 250 nm, as measured by light scattering. In one embodiment, the prostacyclin is treprostinil. In a further embodiment, the particulate composition is in powder or liquid form, and is delivered to the lungs of a patient in need thereof as an aerosol via an inhalation device (e.g., nebulizer), at a rate of about 0.1 to about 1.0 mL/min. In one embodiment, the particulate composition comprises treprostinil, and is in dry powder form. In a further embodiment, the dry powder composition is delivered to the lungs of a patient in need thereof via an inhalation device, e.g., a dry powder inhaler.

In one embodiment, the present invention provides particle composition that incorporates the prostacyclin or analog thereof and the cationic compound, and provides a controlled release of the drug over time thus allowing for a dose frequency to two or three times a day, or less. In one embodiment, the cationic compound is a cationic lipid. The pharmaceutical composition of the invention, in one embodiment, also reduces systemic hemodynamic effects such as changes in blood pressure. Another benefit of the pharmaceutical composition described herein, in one embodiment, is to further reduce acute exposure on nebulization that triggers cough.

In one embodiment, the present invention also provides aerosolized particles that retain or release the prostacyclin or analog thereof over the course of a 6-24 hour period and maximize residence time in the lung (avoid uptake by phagocytic cells and lung surfactant cells) through use of stealth design.

The present invention, in one embodiment, also provides pulmonary hypertension and portopulmonary hypertension patients with an improved prostacyclin composition that is efficacious while improving patient tolerability and compliance with treatment. Certain prostacyclins are indicated for the treatment of pulmonary hypertension, and the compositions provided herein, in one embodiment, reduce dose frequency from 4-times a day for currently approved prostacyclin therapies to 1×, 2× or 3× daily, while significantly reducing the incidence of severe cough, throat irritability, and pain, thus improving tolerability. The pharmaceutical composition described herein, in one embodiment, reduces patient burden and discomfort caused by the currently available pulmonary hypertension medications, for example, pulmonary arterial hypertension medications.

### BRIEF DESCRIPTION OF THE FIGURES

FIG. 1 is a cartoon drawing of three embodiments of the invention. The top drawing shows a solid particle comprising a prostacyclin (e.g., treprostinil) or prostacyclin analog (e.g., trcprostinil), a cationic lipid, and a surfactant (e.g., a PEGylated lipid). The middle image represents a small particle coated with lipid where the complexation is between a cationic compound and prostacylin or prostacylin analog. The cationic compound, in one embodiment, is an inorganic ion or cationic lipid. The bottom image is of a liposome where the prostacyclin or prostacyclin analog is complexed with a cationic compound inside the liposome, and a polymer lipid (surfactant) is part of the surface structure.

FIG. 2 illustrates the chemical structures of representative treprostinil acid and salts, for use with the present invention.

FIG. 3 is a diagram of one embodiment for manufacturing a treprostinil composition of the present invention.

FIG. 4A is a graph of nanoparticle diameter of compositions of the present invention having a fixed ratio of treprostinil:cationic lipid, as a function of squalane concentration.

FIG. 4B is a graph of nanoparticle diameter of compositions of the present invention having a fixed ratio of treprostinil:cationic lipid:PEGylated lipid, as a function of squalane concentration.

FIG. 5 is a graph of particle size as a function of PEGylated-lipid mol %.

FIG. 6A is a graph showing the percent of particle associated treprostinil as a function of cationic lipid content (mol %) used to prepare the respective compositions.

FIG. 6B is a graph of free treprostinil as a function of (i) cationic lipid present in the respective treprostinil composition and (ii) particle charge of each composition.

FIG. 6C is a graph of free treprostinil as a function of (i) cationic lipid present in the respective treprostinil composition and (ii) particle charge of each composition.

FIG. 7 is a graph of the amount of treprostinil from either associated lipid particles or as free treprostinil as a function of dialysis time.

FIGS. 8A-C are graphs of relative cAMP response of CHO-K1-P4 cells (2.5×10⁴ cells/well) as a function of time, in response to 10 μM treprostinil, 7 μM T527 and 5 μM T550 (FIG. 8A), 1 μM treprostinil, T527 and T550 (FIG. 8B) or 0.1 μM treprostinil, T527 and T550 (FIG. 8C) T527-4, as measured by a modified GloSensor assay.

FIG. 9A is a graph of relative cAMP response of CHO-K1-P4 cells (2.5×10⁴ cells/well) as a function of time, in response to free treprostinil (2 μM). T420 (pre-nebulization), T420 (post-nebulization, 2 μM), T471 (pre-nebulization, 2 μM) and T471 (post-nebulization, 2 μM).

FIG. 9B is a graph of relative cAMP response of CHO-K1-P4 cells (2.5×10⁴ cells/well) as a function of time, in response to free treprostinil (2 μM). T441 (pre-nebulization), T441 (post-nebulization, 2 μM), T470 (pre-nebulization, 2 μM) and T470 (post-nebulization, 2 μM).

FIGS. 10A-10D are graphs showing the CHO-K1 cell proliferation inhibition as function of treprostinil concentration. Cells were treated for a 48 hr. period with the respective compositions. T527 (FIG. 10A), T550 (FIG. 10B), T441 (FIG. 10C), T420 (FIG. 10D).

FIGS. 11A-11D are graphs showing NR8383 rat alveolar cell proliferation inhibition a 48 as function of treprostinil concentration. Cells were treated for a 72 hr. period with the respective compositions. T527 (FIG. 11A), T550 (FIG. 11B), T441 (FIG. 11C), T420 (FIG. 11D).

FIG. 12 is a graph of pulmonary arterial pressure (expressed as a percent of hypoxic baseline value) as a function of time, in animals challenged with free treprostinil T527 or T550.

FIG. 13A-13B are graphs of the systemic arterial pressure (expressed as a percent of the baseline hypoxic value) vs. time, in response to animals challenged with PBS, free treprostinil, T527 or T550.

FIG. 14A is a graph of in vivo heart rate (expressed as “BPM” or “beats per minute”) as a function of time in response to animal challenge with PBS, treprostinil, T527 and T550 in an in vivo acute hypoxia rat model of PAH.

FIG. 14B is a graph of in vivo heart rate (expressed as a percent from starting hypoxia value) as a function of time, in response to animal challenge with PBS, treprostinil, T527 and T550 in an in vivo acute hypoxia rat model of PAH. The vertical dashed line marks change in x-axis time increments.

### DETAILED DESCRIPTION OF THE INVENTION

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or there below. Furthermore, the phrases “less than about [a value]” or “greater than about [a value]” should be understood in view of the definition of the term “about” provided herein. The terms “about” and “approximately” are used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

Throughout the present specification, the words “a” or “an” are understood to mean “one or more” unless explicitly stated otherwise. Further, the words “a” or “an” and the phrase “one or more” may be used interchangeably.

The term “treating” includes: (1) preventing or delaying the appearance of clinical symptoms of the state, disorder or condition developing in the subject that may be afflicted with or predisposed to the state, disorder or condition but does not yet experience or display clinical or subclinical symptoms of the state, disorder or condition: (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (e.g., causing regression of the state, disorder or condition or at least one of its clinical or subclinical symptoms). The benefit to a subject to be treated is either statistically significant or at least perceptible to the subject or to the physician.

“Prophylaxis,” as used herein, can mean complete prevention of an infection or disease, or prevention of the development of symptoms of that infection or disease; a delay in the onset of an infection or disease or its symptoms; or a decrease in the severity of a subsequently developed infection or disease or its symptoms.

“Effective amount” means an amount of prostacyclin composition used in the present invention sufficient to result in the desired therapeutic response.

“Liposomal dispersion” refers to a solution or suspension comprising a plurality of liposomes.

An “aerosol,” as used herein, is a gaseous suspension of liquid or dry particles. The aerosol provided herein, in one embodiment, comprises the pharmaceutical composition described herein.

The terms “hydrophobic additive” and “hydrophobic filler” are used interchangeably herein.

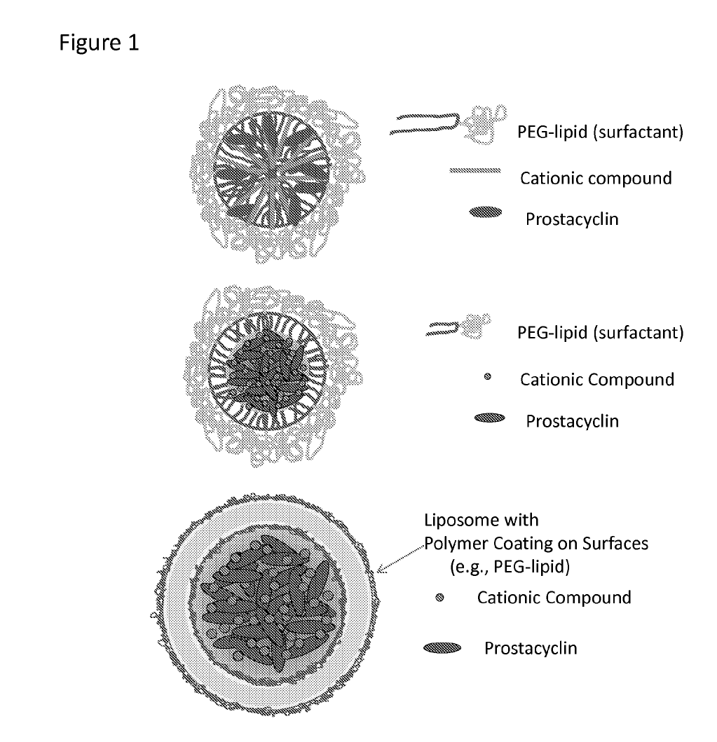

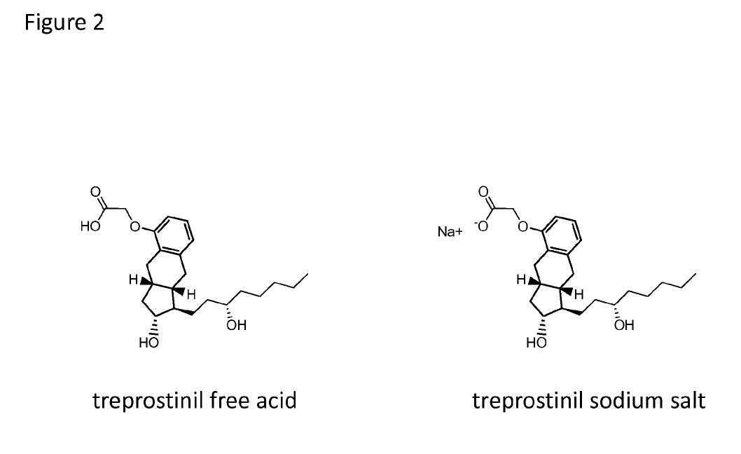

In one aspect, the present invention relates to a pharmaceutical composition comprising a prostacyclin or analog thereof, a cationic compound, and a surfactant. FIG. 1 depicts embodiments of this aspect, where the composition is in the form of a particle, e.g., a colloidal particle or nanoparticle. In particular, FIG. 1 shows a particle comprising a prostacyclin or prostacyclin analog, a cationic compound and a surfactant. The cationic compound, in one embodiment, allows the prostacyclin to be sequestered in particle form. Without wishing to be bound by theory, it is thought that the cationic compound reduces exchange with bulk solution via electrostatic interaction. The cationic compound, in one embodiment, is hydrophobic and interacts electrostatically with the prostacyclin or prostacyclin analog. The surfactant, in one embodiment, provides surface coating of the particle to reduce interaction with biological tissue where exchange of the prostacyclin or prostacyclin analog would be hastened by collision exchange and erosion by interaction with biological materials. Use of a PEGylated lipid as the surfactant, in the compositions provided herein, in one embodiment, minimizes uptake by macrophages.

In one embodiment, the prostacyclin or prostacyclin analog in the pharmaceutical composition is treprostinil. Accordingly, in one embodiment, the pharmaceutical composition comprises treprostinil, a cationic compound, and a surfactant. In another embodiment, the pharmaceutical composition comprises treprostinil, a cationic compound, a surfactant and a hydrophobic additive. In a further embodiment, the pharmaceutical composition is in particle form, for example a micelle particle or a solid nanoparticle. In a further embodiment, the cationic compound is a cationic lipid or an inorganic cation (e.g., a metal cation) (FIG. 1, middle). In even a further embodiment, the cationic compound is multicationic.

The pharmaceutical composition provided herein, in one embodiment, comprises a prostacyclin selected from treprostinil, epoprostenol, iloprost, or analog thereof, for example, a treprostinil, epoprostenol or an iloprost analog. The composition, in one embodiment, comprises a plurality of particles, e.g., nanoparticles. The plurality of particles can comprise solid particles, nanoparticles, solid lipid nanoparticles, micelles, liposomes or proliposomes, or a mixture thereof (FIG. 1). In one embodiment, the pharmaceutical composition is a dispersion comprising a micellar, proliposomal, or liposomal complexed prostacyclin or a prostacyclin encapsulated in a micelle, liposome, or proliposome. A “liposomal complexed prostacyclin” includes embodiments where the prostacyclin (or combination of prostacyclin) is encapsulated in a liposome, and includes any form of prostacyclin composition where at least about 1% by weight of the prostacyclin is associated with the liposome either as part of a complex with a liposome, or as a liposome where the prostacyclin may be in the aqueous phase, in a soluble or precipitated or complexed form, or the hydrophobic bilayer phase or at the interfacial headgroup region of the liposomal bilayer.

In one embodiment, the pharmaceutical composition provided herein comprises a prostacyclin complexed with a cationic compound, where the prostacyclin is present in particle form, for example, as a solid nanoparticle, a colloidal particle, a micelle or a liposome. In one embodiment, at least about 1% by weight of the prostacyclin is associated with the cationic compound, e.g., a cationic lipid, for example as a part of a complex or a nanoparticle.

In one embodiment, the composition is administered to a patient in need thereof via nebulization, for example for the treatment of pulmonary hypertension or portopulmonary hypertension, and prior to nebulization of the composition, at least about 5%, at least about 10%, at least about 20%, at least about 25%, at least about 50%, at least about 75%, at least about 80%, at least about 85%, at least about 90% or at least about 95% of the prostacyclin or prostacyclin analog in the composition is associated with the cationic compound in particle form. Association, in one embodiment, is measured by separation through a filter where cationic compound and cationic compound-associated drug is retained (i.e., in the retentate) and free drug is in the filtrate.

In one embodiment, the prostacyclin is associated with the cationic compound and form a particle, for example a colloidal particle or nanoparticle. In another embodiment, the prostacyclin associated with the cationic compound as a micelle or as a liposome (FIG. 1, bottom). In the case of a liposome, the prostacyclin may be in the aqueous phase or the hydrophobic bilayer phase or at the interfacial headgroup region of the liposomal bilayer.

In another embodiment, the composition provided herein is a micellar dispersion or a nanoparticle composition comprising a prostacyclin or prostacyclin analog, a cationic compound and a surfactant. In a further embodiment, the micellar dispersion or nanoparticle composition comprises a hydrophobic additive, e.g., squalane. For example, in one embodiment, the composition comprises treprostinil, a cationic lipid, a PEGylated lipid and squalane. In one embodiment, the composition comprises prostacyclin and the cationic compound, e.g., the cationic lipid in a micelle or a nanoparticle. The micellar dispersion or nanoparticle composition, in one embodiment, has at least about 1% by weight of the prostacyclin associated with the cationic compound, for example electrostatically associated.

In one embodiment, the fine particle fraction (FPF) of the composition post nebulization, i.e., the aerosolized pharmaceutical composition, is about 50%, or about 55%, or about 60%, or about 65%, or about 70%, or about 75%, as measured by NGI or ACI. In a further embodiment, the FPF of the aerosol is greater than or equal to about 64%, as measured by the ACI, greater than or equal to about 70%, as measured by the ACI, greater than or equal to about 51%, as measured by the NGI, or greater than or equal to about 60%, as measured by the NGI.

The compositions, systems, and methods provided herein, in one embodiment, comprise a prostacyclin or analog thereof, cationic compound and a surfactant. In one embodiment, the composition is in particle form, for example a micelle particle or a solid lipid nanoparticle. In one embodiment, the cationic compound is a lipid. In one embodiment, the composition comprises a lipid-encapsulated or lipid-associated prostacyclin or analog thereof, for example a solid lipid nanoparticle. The lipids used in the pharmaceutical compositions of the present invention can be synthetic, semi-synthetic or naturally-occurring lipids, including phospholipids, tocopherols, tocopherol derivatives, sterols, sterol derivatives, and fatty acids.

The cationic compound in the pharmaceutical composition provided herein may be monocationic or multicationic. In one embodiment, the cationic compound is net cationic, i.e., the compound has both positive and negative charges with a net positive charge. Examples of the cationic compound include, but are not limited to, a cationic lipid, alkyl-ammonium, alkyl-polyammonium, linear polyamine, linear polyethylenimine, branched polyethylenimine, poly-L-lysine, trimethyl-poly-glucosamine, an inorganic ion, a metal ion, a multivalent inorganic ion, or multivalent metal ion. In a further embodiment, the cationic compound may be dioctadecyldimethyl ammonium bromide (diC18dMA), dimethyldihexadecylammonium chloride, N-[1-(2,3-dioleoyloxy)propyl]-N,N,N-trimethylammonium methyl sulfate (DOTAP), N-[1-(2,3-dioleyloxyl)propyl]-N,N,N-trimethylammonium chloride (DOTMA), 1,2-distearoyl-3-(trimethylammonio)propane chloride (DSTAP), dimyristoyltrimethylammonium propane (DMTAP), or dioctadecyldimethylammonium bromide (DODAB). The cationic compound may also be N,N′-dihexadecyl-1,2-ethanediamine, tetracthylhexadecane-1,16-diamine, or hexadecane-1,16-bis(trimethylammonium bromide). In one embodiment, the cationic compound is a metal cation such as aluminum, magnesium, beryllium, strontium, barium, or calcium. Other multivalent metals may also be used. In one embodiment, the cationic compound is dioctadecyldimethyl ammonium bromide (dc18dMA).

In one embodiment, the at least one cationic compound is a cationic lipid (i.e., a positively charged lipid). The cationic lipid used can include ammonium salts of fatty acids, phospholipids and glycerides, and sterol derivatives. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), and 1,2-bis(oleoyloxy)-3-(trimethylammonio)propane (DOTAP).

In one embodiment, the at least one surfactant in the composition is neutral, nonionic, cationic, or anionic. The surfactant, in one embodiment, is amphiphilic, a PEGylated lipid or a block copolymer. In a further embodiment, the at least one surfactant comprises at least one anionic surfactant.

The surfactant, in one embodiment, is a PEGylated lipid. In a further embodiment, the PEGylated lipid comprises PEG400, PEG500, PEG1000, PEG2000, PEG3000, PEG4000, or PEG5000. In a further embodiment the lipid component of the PEGylated lipid comprises PEG covalently linked to dimyristoyl phosphatidylethanolamine (DMPE), dipalmitoyl phosphoethanolamine (DPPE), distearoylphosphatidylethanolamine (DSPE), dimyristoylglycerol glycerol (DMG), diphosphatidylglycerol (DPG), disteraroylglycerol (DSG) or cholesterol. Depending on its molecular weight (MW), PEG is also referred to in the art as polyethylene oxide (PEO) or polyoxyethylene (POE). The PEGylated lipid can include a branched or unbranched PEG molecule, and is not limited by a particular PEG MW.

For example, the PEGylated lipid (PEG-lipid), in one embodiment, comprises a PEG molecule having a molecular weight of 300 g/mol, 400 g/mol, 500 g/mol, 1000 g/mol, 1500 g/mol, 2000 g/mol, 2500 g/mol, 3000 g/mol, 3500 g/mol, 4000 g/mol, 4500 g/mol, 5000 g/mol or 10,000 g/mol. In one embodiment, the PEG has a MW of 1000 g/mol or 2000 g/mol.

The lipid component of the PEGylated lipid (or “PEG-lipid”), can have a net-charge (e.g., cationic or anionic), or can be net-neutral. The lipids used in the PEGylated lipid component of the present invention can be synthetic, semi-synthetic or naturally-occurring lipid, including a phospholipid, a sphingolipid, a glycolipid, a ceramide, a tocopherol, a steroid, a fatty acid, or a glycoprotein such as albumin. In one embodiment, the lipid is cholesterol. In another embodiment, the lipid is a phospholipid. Phospholipids include, but are not limited to phosphatidylcholine (PC), phosphatidylglycerol (PG), phosphatidylinositol (PI), phosphatidylscrine (PS), phosphatidylethanolamine (PE), and phosphatidic acid (PA). In one embodiment, the phospholipid is an egg phospholipid, a soya phospholipid or a hydrogenated egg and soya phospholipid. In one embodiment, the PEGylated lipid comprises a phospholipid. In a further embodiment, the phospholipid comprises ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid can be made up of fatty acids of different chain lengths and different degrees of unsaturation. In particular, in one embodiment, the PEGylated lipid of the prostacyclin composition provided herein comprises distearoylphosphoethanolamine (DSPE), dipalmitoylphosphatidylcholine (DPPC), dioleoylphosphatidylcholine (DOPC) dimyristoyl phosphatidylethanolamine (DMPE), dipalmitoylphosphoethanolamine (DPPE), distearoylphosphatidylethanolamine (DSPE), dimyristoylglycerol (DMG), diphosphatidylglycerol (DPG) or disteraroylglycerol (DSG).

Other examples of lipids for use in the compositions comprising PEGylated lipids disclosed herein include dimyristoylphosphatidylcholine (DMPC), dimyristoyiphosphatidylglycerol (DMPG), dipalmitoylphosphatidylglycerol (DPPG), distearoylphosphatidylcholine (DSPC), distearoylphosphatidylglycerol (DSPG), dioleylphosphatidylethanolamine (DOPE), and mixed phospholipids such as palmitoylstearoylphosphatidylcholine (PSPC) and palmitoylstearoylphosphatidylglycerol (PSPG), triacylglycerol, diacylglycerol, ceramide, sphingosine, sphingomyelin and single acylated phospholipids such as mono-oleoyl-phosphatidylethanolamine (MOPE). In another embodiment lipid portion of the PEGylated lipid comprises an ammonium salt of a fatty acid, a phospholipid, a glyceride, a phospholipid and glyceride, a sterol (e.g., cholesterol), phosphatidylglycerol (PG), phosphatidic acid (PA), a phosphotidylcholine (PC), a phosphatidylinositol (PI), a phosphatidylserine (PS), or a combination thereof. The fatty acid, in one embodiment, comprises fatty acids of carbon chain lengths of 12 to 26 carbon atoms that are either saturated or unsaturated. Some specific examples include: myristylamine, palmitylaminc, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9 (Z)-octadecenyloxy)-prop-1-yl-N,N,N-trimethylammonium chloride (DOTMA) and 1,2-bis(oleoyloxy)-3-(trimethylammonio)propane (DOTAP). Examples of sterols for use in the compositions provided herein include cholesterol and ergosterol. Examples of PGs, PAs, PIs, PCs and PSs for use in the compositions provided herein include DMPG, DPPG, DSPG, DMPA, DPPA, DSPA, DMPI, DPPI, DSPI, DMPS, DPPS and DSPS, DSPC, DPPG, DMPC, DOPC, egg PC and soya PC.

In one embodiment, the PEGylated lipid is cholesterol-PEG2000, DSPE-PEG1000 or DSG-PEG2000.

Other examples of surfactants for use in the compositions of the invention include, without limitation, polyoxyethyleneglycol-lipid, polyoxypropyleneglycol-lipid, glucoside-lipid, glycerol-lipid, or polysorbate-lipid. In one embodiment, the surfactant is an anionic lipid (negatively charged lipid). The negatively-charged lipids which can be used include phosphatidyl-glycerols (PGs), phosphatidic acids (PAs), phosphatidylinositols (PIs) and the phosphatidyl serines (PSs). Examples include DMPG, DPPG, DSPG, DMPA, DSPA, DPPS. DSPS, DPPA, DMPI, DPPI, DSPI and DMPS.

In another embodiment of the invention, the pharmaceutical compositions provided herein further comprise a hydrophobic additive. In one embodiment, the hydrophobic additive may be a hydrocarbon, a terpene or a hydrophobic lipid. In a further embodiment, the hydrophobic additive may be without limitation cholesteryl acetate, ethyl stearate, palmitate, myristate, palmityl palmitate, tocopheryl acetate, a monoglyceride, a diglyceride, a triglyceride like palmitate, myristate, dodecanoate, decanoate, octanoate, or squalane.

In one embodiment, the pharmaceutical composition of the invention, comprises squalane as a hydrophobic additive. In one embodiment, the mole range of squalane is from about 0.1 mol % to about 28 mol % of the composition. In another embodiment, the squalane concentration is 25 mol %.

In yet another embodiment of the invention, the pharmaceutical composition comprising a hydrophobic additive may also include the surfactant herein described. In this embodiment, the pharmaceutical composition may comprise a polyoxyethyleneglycol-phospholipid. A suitable polyoxyethyleneglycol-phospholipid used for this embodiment of the invention is polyoxyethyleneglycol-cholesterol. In another embodiment, the pharmaceutical composition comprising the hydrophobic additive may also include a polyoxyethyleneglycol-lipid. The polyoxyethyleneglycol-lipid may be without limitation distearoylphosphatidylethanolamine-polyoxyethyleneglycol or disteraroylglycerol-polyoxyethyleneglycol.

In accordance with one embodiment of the invention, the hydrophobic additive may be present in the composition at 30%-50 mol %, for example, 35-45 mol %. In even a further embodiment, the hydrophobic additive is present in the composition at 40 mol %.

In one embodiment, the hydrophobic additive (e.g., an additive that is at least partially hydrophobic), when present in a composition comprising the prostacyclin compound and the cationic compound, is a hydrocarbon, a terpene compound or a hydrophobic lipid (e.g., tocopherol, tocopherol acetate, sterol, sterol ester, alkyl ester, vitamin A acetate, a triglyceride, a phospholipid).

The terpene compound (hydrophobic additive), in one embodiment, is a hydrocarbon (e.g., isoprene or squalene). In another embodiment, the terpene compound is a hemiterpene (C₅H₈), monoterpene (C₁₀H₁₆), sesquiterpene (C₁₅H₂₄), diterpene (C₂₀H₃₂) (e.g., cafestol, kahweol, cembrene, taxadiene), sesterterpene (C₂H₄₀), triterpene (C₃₀H₄₈), sesquaterpene (C₃₅H₅₆), tetraterpene (C₄₀H₆₄), polyterpene (e.g., a polyisoprene with trans double bonds) or a norisoprenoid (e.g., 3-oxo-α-ionol, 7,8-dihydroionone derivatives). The terpene compound, in another embodiment, is selected from one of the compounds provided in Table 1, below.

TABLE 1
Terpene hydrophobic additives amenable for use in the compositions of the present invention.

| Name | Formula |
|---|---|
| Isoprene | 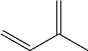 |
| Limonene | 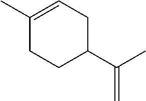 |
| humulene | 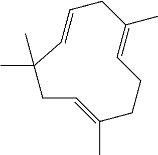 |
| farnasene | 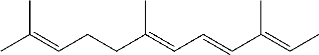 |
| squalene | 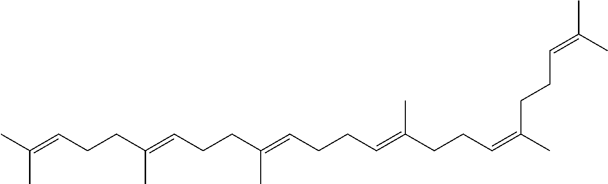 [SMILES: CC(C)=CCC/C(C)=C\CC/C(C)=C/CC/C=C(\C)CC/C=C(\C)CCC=C(C)C] |
| squalane | 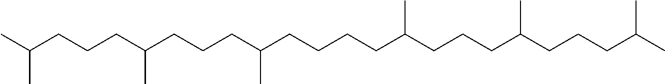 [SMILES: CC(C)CCCC(C)CCCC(C)CCCCC(C)CCCC(C)CCCC(C)C] |

In one embodiment, the prostacyclin composition provided herein is in particle form. Accordingly, in one embodiment, the pharmaceutical composition provided herein comprises a plurality of particles comprising the prostacyclin or analog thereof and the cationic compound. In a further embodiment, the surfactant is associated with at least one of the plurality of particles in the composition.

The plurality of particles in the pharmaceutical composition, in one embodiment, comprises solid colloidal particles, polymer-lipid hybrid nanoparticles (Vieira and Carmona-Ribeiro (2008). Journal of Nanobiotechnology 6:1-13, incorporated by reference in its entirety), nanostructured lipid carriers, polymeric microspheres (Liu et al. (2000). J Pharm Pharmacol, 53:1-12, incorporated by reference in its entirety), nanoparticles, micelles, liposomes, solid lipid nanoparticles (Wong et al. (2004). J Pharm Sci, 93:1993-2004, incorporated by reference in its entirety), or a combination thereof.

As provided above, in one embodiment, the pharmaceutical composition provided herein comprises a plurality of solid particles comprising at least one cationic compound and a prostacyclin or analog thereof. In one embodiment, the cationic compound forms the core of a particle of the invention, and the at least one surfactant stabilizes the cationic compound (FIG. 1). In a further embodiment, the at least one surfactant is a PEGylated lipid.

In one embodiment, the pharmaceutical composition provided herein comprises a plurality of solid lipid nanoparticles (SLNs) comprising a solid lipid core stabilized by a surfactant. In one embodiment, the core lipid is a cationic lipid, for example, one of the cationic lipids described above. In a further embodiment, the prostacyclin or analog thereof associates at the core of the particle, at the outer layer of the particle, or a combination thereof.

In one embodiment, the pharmaceutical composition provided herein comprises a plurality of solid polymer nanoparticles comprising a cationic polymer, prostacyclin or an analog thereof and a surfactant polymer (e.g., a PEGylated lipid). In a further embodiment, the plurality of particles are formed by electrostatic interactions between the at least one cationic polymer and the at least one surfactant polymer (see, e.g., Vieira and Carmona-Ribeiro (2008). Journal of Nanobiotechnology 6:1-13, incorporated by reference in its entirety). In one embodiment, the prostacyclin or analog thereof associates with the particle via electrostatic interaction or hydrophobic interaction, or a combination thereof.

The prostacyclin or analog thereof, cationic compound and surfactant, in one embodiment, self assemble into a plurality of particles. For example, certain lipids such as dioctadecyldimethylammonium bromide (DODAB) and sodium dihxadecylphosphate (DHP) self-assemble in aqueous solution depending on the procedure for dispersing the lipid.

In one embodiment, at least about 1% or at least 10%, or at least 25%, or at least 50% or at least 75% or at least 90% of the composition is in particle form, either as a single particle or a plurality of particles. The average diameter of the plurality of particles in the composition, prior to administration, in one embodiment, is about 500 nm or less, as measured by light scattering. In another embodiment, the average diameter of the particle(s) in the composition is about 400 nm or less, or about 300 nm or less, or about 200 nm or less, or about 100 nm or less, or about 50 nm or less, as measured by light scattering. In another embodiment, the average diameter of the particle(s) in the composition is about 100 nm to about 500 nm, or about 150 nm to about 500 nm, or about 200 nm to about 500 nm, or about 250 nm to about 500 nm, or about 300 nm to about 500 nm, or about 350 nm to about 500 nm, as measured by light scattering. In one embodiment, the particle or plurality of particles is a solid particle or a plurality of solid particles (e.g., solid lipid nanoparticles). In one embodiment, the mean diameter of the plurality of particles in the composition is about 10 to about 100 nm, about 50 nm to about 100 nm, about 100 nm to about 200 nm, about 200 nm to about 300 nm, about 210 nm to about 290 nm, about 220 nm to about 280 nm, about 230 nm to about 280 nm, about 240 nm to about 280 nm, about 250 nm to about 280 nm or about 260 nm to about 280 nm, as measured by light scattering. In a further embodiment, the particle or particle(s) is a solid lipid nanoparticle or a plurality of solid lipid nanoparticles, or a micelle or a plurality of micelle(s).

In another embodiment, the plurality of particles is a plurality of micelles or liposomes. Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer) or a combination thereof. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes can be produced by a variety of methods (see, e.g., Cullis et al. (1987)). In one embodiment, one or more of the methods described in U.S. Patent Application Publication No. 2008/0089927 are used herein to produce the prostacyclin encapsulated lipid compositions (liposomal dispersion). The disclosure of U.S. Patent Application Publication No. 2008/0089927 is incorporated by reference in its entirety for all purposes. For example, in one embodiment, at least one lipid and a prostacyclin are mixed with a coacervate (i.e., a separate liquid phase) to form the liposome composition. The coacervate can be formed prior to mixing with the lipid, during mixing with the lipid or after mixing with the lipid. Additionally, the coacervate can be a coacervate of the active agent.

In one embodiment, the liposomal dispersion is formed by dissolving one or more lipids in an organic solvent forming a lipid solution, and the prostacyclin coacervate forms from mixing an aqueous solution of the prostacyclin with the lipid solution. In a further embodiment, the organic solvent is ethanol. In even a further embodiment, the one or more lipids comprise a phospholipid and a sterol.

In one embodiment, liposomes are produced by sonication, extrusion, homogenization, swelling, electroformation, inverted emulsion or a reverse evaporation method. Bangham's procedure (J. Mol. Biol. (1965), incorporated by reference herein in its entirety) produces ordinary multilamellar vesicles (MLVs). Lenk et al. (U.S. Pat. Nos. 4,522,803, 5,030,453 and 5,169,637), Fountain et al. (U.S. Pat. No. 4,588,578, incorporated by reference herein in its entirety) and Cullis et al. (U.S. Pat. No. 4,975,282, incorporated by reference herein in its entirety) disclose methods for producing multilamellar liposomes having substantially equal interlamellar solute distribution in each of their aqueous compartments. Paphadjopoulos et. al., U.S. Pat. No. 4,235,871, incorporated by reference herein in its entirety, discloses preparation of oligolamellar liposomes by reverse phase evaporation. Each of the methods is amenable for use with the present invention. Each of the patents disclosed in this paragraph is incorporated by reference herein for all purposes.

Unilamellar vesicles can be produced from MLVs by a number of techniques, for example, the extrusion techniques of U.S. Pat. No. 5,008,050 and U.S. Pat. No. 5,059,421, each incorporated by reference herein for all purposes. Sonication and homogenization cab be so used to produce smaller unilamellar liposomes from larger liposomes (see, for example, Paphadjopoulos et al. (1968); Deamer and Uster (1983); and Chapman et al. (1968), each of which is incorporated by reference herein in their entireties).

The liposome preparation of Bangham et al. (J. Mol. Biol. 13, 1965, pp. 238-252) involves suspending phospholipids in an organic solvent which is then evaporated to dryness leaving a phospholipid film on the reaction vessel. Next, an appropriate amount of aqueous phase is added, the 60 mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys. Acta. 135, 1967, pp. 624-638, incorporated by reference herein in its entirety), and large unilamellar vesicles.

Techniques for producing large unilamellar vesicles (LUVs), such as, reverse phase evaporation, infusion procedures, and detergent dilution, can be used to produce liposomes for use in the pharmaceutical compositions provided herein. A review of these and other methods for producing liposomes may be found in the text Liposomes, Marc Ostro, ed., Marcel Dekker, Inc., New York, 1983, Chapter 1, which is incorporated herein by reference. See also Szoka, Jr. et al., (Ann. Rev. Biophys. Bioeng. 9, 1980, p. 467, incorporated by reference herein in its entirety), which is also incorporated herein by reference in its entirety for all purposes.

Other techniques for making liposomes include those that form reverse-phase evaporation vesicles (REV), U.S. Pat. No. 4,235,871. Another class of liposomes that may be used is characterized as having substantially equal lamellar solute distribution. This class of liposomes is denominated as stable plurilamellar vesicles (SPLV) as defined in U.S. Pat. No. 4,522,803, incorporated by reference herein in its entirety, and includes monophasic vesicles as described in U.S. Pat. No. 4,588,578, incorporated by reference herein in its entirety, and frozen and thawed multilamellar vesicles (FATMLV) as described above.

A variety of sterols and their water soluble derivatives such as cholesterol hemisuccinate have been used to form liposomes; see, e.g., U.S. Pat. No. 4,721,612, incorporated by reference herein in its entirety. Mayhew et al., PCT Publication No. WO 1985/00968, incorporated by reference herein in its entirety, describes a method for reducing the toxicity of drugs by encapsulating them in liposomes comprising alpha-tocopherol and certain derivatives thereof. Also, a variety of tocopherols and their water soluble derivatives have been used to form liposomes, see PCT Publication No. 87/02219, incorporated by reference herein for all purposes.

In some embodiments, the pharmaceutical compositions herein described may have a surfactant comprising individual components in a molecular ratio of 1:9, 2:8, 3:7, 4:6, 5:5, 6:4, 7:3, 8:2, or 9:1. For example, cholesterol-PEG, DSG-PEG, DSPE-PEG in one embodiment, are in a mol ratio of about 1:1, about 1:9, and about 1:9, respectively. In other embodiments, surfactants such as polyoxyethyleneglycol-lipid or polyoxyethyleneglycol-phospholipid are in a mol ratio of about 1:1 or about 1:9.

The pharmaceutical composition provided herein comprises a prostacyclin or analog thereof, a cationic compound and a surfactant. The cationic compound and surfactant to prostacyclin (or prostacyclin analog) weight ratio in the pharmaceutical composition provided herein, in one embodiment, is 10 to 1 or less, 9 to 1 or less, 8 to 1 or less, 7 to 1 or less, 5 to 1 or less, 3 to 1 or less, 2.5 to 1 or less, 2 to 1 or less, 1.5 to 1 or less, 1 to 1 or less, 0.5 to 1 or less, or 0.1 to 1 or less.

The cationic compound to prostacyclin (or prostacyclin analog) weight ratio in the pharmaceutical compositions provided herein, in one embodiment, is 10 to 1 or less, 9 to 1 or less, 8 to 1 or less, 7 to 1 or less, 5 to 1 or less, 3 to 1 or less, 2.5 to 1 or less, 2 to 1 or less, 1.5 to 1 or less, 1 to 1 or less, 0.5 to 1 or less, or 0.1 to 1 or less.

In one embodiment, the cationic compound is a cationic lipid and the cationic lipid to prostacyclin (or prostacyclin analog) weight ratio in the pharmaceutical compositions provided herein is 10 to 1 or less, 9 to 1 or less, 8 to 1 or less, 7 to 1 or less, 5 to 1 or less, 3 to 1 or less, 2.5 to 1 or less, 2 to 1 or less, 1.5 to 1 or less, 1 to 1 or less, 0.5 to 1 or less, or 0.1 to 1 or less.

In another embodiment, the cationic compound is diC18dMA and the prostacyclin is treprostinil. In a further embodiment, the cationic lipid to treprostinil weight ratio in the pharmaceutical compositions provided herein is 10 to 1 or less, 9 to 1 or less, 8 to 1 or less, 7 to 1 or less, 5 to 1 or less, 3 to 1 or less, 2.5 to 1 or less, 2 to 1 or less, 1.5 to 1 or less, 1 to 1 or less, 0.5 to 1 or less, or 0.1 to 1 or less.

In one embodiment, the compositions provided herein further comprise one or more pharmaceutical excipients, or other additives. Such excipients or additives may include one or more stabilizing polyols, e.g., higher polysaccharides/polymers (for promoting controlled release), magnesium stearate, leucine and/or trileucine (as lubricants), and phospholipids and/or surfactants. Blowing agents, e.g., volatile salts such as ammonium carbonate, formic acid, etc. may also be included in the feedstock to produce reduced density particles in the present spray dried powders.

Spray aids may also be employed with the present compositions or systems. Such spray aids may reduce the viscosity and/or improve the fluid mechanical characteristics of the present compositions during the spray drying process. Spray aids may include maltodextrin, lactose, gelatin, talc, triethylcitrate, and mixtures thereof. Such spray aids may be present in the compositions in amounts ranging from about 1 wt % to about 15 wt % (e.g., about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, or any other value or range of values therein). In certain embodiments, the spray aid is maltodextrin, and the amount of maltodextrin in the composition is about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %. In other embodiments, the spray aid is lactose, and the amount of lactose in the composition is about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %. In still other embodiments, the spray aid is gelatin, and the amount of gelatin in the composition is about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %.

In another aspect of the invention, a method for treating pulmonary hypertension (PH) is provided. The World Health Organization (WHO) has classified PH into five groups. Group I PH includes pulmonary arterial hypertension (PAH), idiopathic pulmonary arterial hypertension (IPAH), familial pulmonary arterial hypertension (FPAH), and pulmonary arterial hypertension associated with other diseases (APAH). For example, pulmonary arterial hypertension associated with collagen vascular disease (e.g., scleroderma), congenital shunts between the systemic and pulmonary circulation, portal hypertension and/or HIV infection are included in group I PH. Group II PH includes pulmonary hypertension associated with left heart disease, e.g., atrial or ventricular disease, or valvular disease (e.g., mitral stenosis). WHO group III pulmonary hypertension is characterized as pulmonary hypertension associated with lung diseases, e.g., chronic obstructive pulmonary disease (COPD), interstitial lung disease (ILD), and/or hypoxemia. Group IV pulmonary hypertension is pulmonary hypertension due to chronic thrombotic and/or embolic disease. Group IV PH is also referred to as chronic thromboembolic pulmonary hypertension. Group IV PH patients experience blocked or narrowed blood vessels due to blood clots. Group V PH is the “miscellaneous” category, and includes PH caused by blood disorders (e.g., polycythemia vera, essential thrombocythemia), systemic disorders (e.g., sarcoidosis, vasculitis) and/or metabolic disorders (e.g., thyroid disease, glycogen storage disease).

For example, the methods provided herein can be used to treat group I (i.e., pulmonary arterial hypertension or PAH), group II, group III, group IV or group V PH patients. In one embodiment of the method for treating PH, a method of treating pulmonary arterial hypertension (PAH). In another embodiment, a method for treating chronic thromboembolic pulmonary hypertension patient is provided. In one embodiment, the method comprises administering to a patient in need thereof an effective amount of one of the prostacyclin compositions described herein. In a further embodiment, administration is to the patient via a pulmonary (inhalation), subcutaneous or intravenous route. The compositions of the present invention may be administered alone, or can be co-administered or sequentially administered with other immunological, antigenic, vaccine, or therapeutic compositions.

In one embodiment, the patient in need of treatment is a Class I PAH patient, class II PAH patient, class III PAH patient or class IV PAH patient. Class I PAH patients do not have a limitation of physical activity, as ordinary physical activity does not cause undue dyspnoea or fatigue, chest pain, or near syncope. Treatment is not needed for class I PAH patients. Class II PAH patients have a slight limitation on physical activity. These patients are comfortable at rest, but ordinary physical activity causes undue dyspnoea or fatigue, chest pain or near syncope. Class III PAH patients have a marked limitation of physical activity. Although comfortable at rest, class III PAH patients experience undue dyspnoea or fatigue, chest pain or near syncope as a result of less than ordinary physical activity. Class IV PAH patients are unable to carry out any physical activity without symptoms. Class IV PAH patients might experience dyspnoea and/or fatigue at rest, and discomfort is increased by any physical activity. Signs of right heart failure are often manifested by class IV PAH patients.

In another aspect of the invention, a method for treating portopulmonary hypertension (PPH) is provided. In one embodiment, the method comprises administering to a patient in need thereof an effective amount of one of the prostacyclin compositions described herein. In a further embodiment, administration is to the patient via a pulmonary (inhalation), subcutaneous or intravenous route.

As provided above, the compositions of the present invention can be delivered to a patient in need thereof via inhalation, i.e., with an inhalation device. An “inhalation device” is a device that is used to deliver a pharmaceutical composition to the lungs of a patient. Inhalation devices include nebulizers and inhalers, e.g., a metered dose inhaler or a dry powder inhaler. A dry powder or a liquid can be delivered to the lungs of a patient by an inhalation device. A “nebulizer” is one type of inhalation device, and is a device that converts a liquid into an aerosol of a size that can be inhaled into the respiratory tract. Pneumonic, ultrasonic, electronic nebulizers, e.g., passive electronic mesh nebulizers, active electronic mesh nebulizers and vibrating mesh nebulizers are amenable for use with the invention if the particular nebulizer emits an aerosol with the required properties, and at the required output rate.

The process of pneumatically converting a bulk liquid into small droplets is called atomization. The operation of a pneumatic nebulizer requires a pressurized gas supply as the driving force for liquid atomization. Ultrasonic nebulizers use electricity introduced by a piezoelectric element in the liquid reservoir to convert a liquid into respirable droplets. Various types of nebulizers are described in Respiratory Care, Vol. 45, No. 6, pp. 609-622 (2000), the disclosure of which is incorporated herein by reference in its entirety for all purposes.

Methods for administering treprostinil and analogs thereof for treatment of pulmonary hypertension have been described in U.S. Pat. Nos. 5,153,222; 6,521,212; 7,544,713 and U.S. Patent Application Publication No. 2010/0076083, the disclosure of each are incorporated by reference in their entireties for all purposes.

In one embodiment, administration of an effective amount of a prostacyclin composition of the present invention for the treatment of pulmonary hypertension (PH), pulmonary arterial hypertension (PAH) or portopulmonary hypertension (PPH) by inhalation, subcutaneous or intravenous administration results in a decreased number of side effects, or a reduced severity of one or more side effects (also referred to herein as “adverse events”), compared to the administration of an effective amount of treprostinil, when an effective amount of treprostinil is administered by subcutaneously, intravenously or by inhalation. For example, in one embodiment, a PH, PAH or PPH patient experiences a reduced severity and/or frequency in cough or a reduced cough response when administered a prostacyclin compound or composition of the invention via inhalation (e.g., via nebulization, a dry powder inhaler, or via a metered dose inhaler), compared to the severity and/or frequency of cough or cough response elicited by inhalation administration of treprostinil to the patient.

In another embodiment, intravenous, subcutaneous or inhalation administration of an effective amount of the prostacyclin compound or composition of the invention, compared to subcutaneous, intravenous or inhalation administration of treprostinil, results in a reduced severity of one or more of the following adverse events, or a decreased occurrence of one or more of the following adverse events: headache, throat irritation/pharyngolaryngeal pain, nausea, flushing and/or syncope.

In another embodiment, intravenous, subcutaneous or inhalation administration of an effective amount of the prostacyclin composition of the invention, for the treatment of PH, PAH or PPH, compared to subcutaneous, intravenous or inhalation administration of treprostinil, results in a reduced severity of a systemic adverse events, or a decreased occurrence of a systemic adverse event.

Without wishing to be bound by theory, it is believed that the improved adverse event profile of the prostacyclin compositions of the invention exhibited patients, as compared to treprostinil, results in improved compliance of the patients.

In one embodiment, the prostacyclin compositions of the present invention are administered on a less frequent basis, as compared to currently approved therapies for PH, PAH (e.g., Tyvaso®, Remodulin®) or PPH, while still achieving a substantially equivalent or better therapeutic response. The therapeutic response of the patient, in one embodiment, is a reduction in the pulmonary vascular resistance index (PVRI) from pretreatment value, a reduction in mean pulmonary artery pressure from pretreatment value, an increase in the hypoxemia score from pretreatment value, a decrease in the oxygenation index from pretreatment values, improved right heart function, as compared to pretreatment or improved exercise capacity (e.g., as measured by the six-minute walk test) compared to pretreatment. The therapeutic response, in one embodiment, is an improvement of at least 10%, at least 20%, at least 30%, at least 40% or at least 50%, as compared to pretreatment values. In another embodiment, the therapeutic response is an improvement of about 10% to about 70%, about 10% to about 60%, about 10% to about 50%, about 10% to about 40%, about 10% to about 30%, about 10% to about 20%, about 20% to about 70%, about 20% to about 60% or about 10% to about 50%, as compared to pretreatment levels.

Without wishing to be bound by theory, the less frequent administration of the compounds and compositions of the invention allows for improved patient compliance, as compared to the compliance of patients being administered a different PH, PAH or PPH treatment (e.g., treprostinil—Tyvaso®, Remodulin®).

In another embodiment, the prostacyclin composition is administered via a nebulizer to a patient in need of PH, PAH or PPH treatment. The administration occurs in one embodiment, once daily, twice daily, three times daily or once every other day.

In one embodiment, a composition or compound of the present invention is administered via a dry powder inhaler (DPI) to a patient in need of PH, PAH or PPH treatment. The patient, in one embodiment, is administered the prostacyclin composition of the invention once daily, twice daily or three times daily. In one embodiment, the administration is with food. In one embodiment, each administration comprises 1 to 5 doses (puffs) from a DPI, for example 1 dose (1 puff), 2 dose (2 puffs), 3 doses (3 puffs), 4 doses (4 puffs) or 5 doses (5 puffs). The DPI, in one embodiment, is small and transportable by the patient.

In another embodiment, the prostacyclin composition administered to a patient in need thereof via a pulmonary route by the PH, PAH or PAH treatment methods described herein provides a greater pulmonary elimination half-life (t_{1,2}) of the prostacyclin compound, compared to the t_{1/2} of free prostacyclin, when the free prostacyclin (e.g., free treprostinil) is administered via a pulmonary route (e.g., by nebulization, dry powder inhaler, or a metered dose inhaler) to the patient in need of PH, PAH or PPH treatment.

In another embodiment, the prostacyclin compound administered to a patient in need thereof, via the PH, PAH or PPH treatment methods described herein provides a greater systemic half-life (t_{1/2}) of the prostacyclin compound, compared to the systemic elimination half-life (t_{1/2}) of treprostinil, when the free prostacyclin (e.g., free treprostinil) is administered to the patient. In a further embodiment, administration of the prostacyclin composition and treprostinil comprises either subcutaneous or intravenous administration.

In another embodiment, the prostacyclin compound administered to a patient in need of PH, PAH or PPH treatment provides a greater mean pulmonary Cₘₐₓ and/or lower plasma Cₘₐₓ of the prostacyclin compound for the patient, compared to the respective pulmonary or plasma Cₘₐₓ of treprostinil, when the free prostacyclin (e.g., free treprostinil) is administered to the patient. In a further embodiment, administration of the prostacyclin composition and the free prostacyclin comprises intravenous administration.

In another embodiment, the prostacyclin composition administered to a patient in need of PH, PAH or PPH treatment provides a greater mean pulmonary or plasma area under the curve (AUC₀₋ₜ) of the prostacyclin compound, compared to the mean pulmonary or plasma area under the curve (AUC₀₋ₜ) of the prostacyclin compound, when the free prostacyclin (e.g., free treprostinil) is administered to the patient. In yet another embodiment, the prostacyclin composition administered to a patient in need thereof provides a greater pulmonary or plasma time to peak concentration (tₘₐₓ) of the prostacyclin compound, compared to the pulmonary or plasma time to peak concentration (tₘₐₓ) of the prostacyclin compound, when the free prostacyclin (e.g., free treprostinil) is administered to the patient.

As provided above, the prostacyclin compounds and compositions of the present invention can be delivered to a patient in need thereof via pulmonary, intravenous or subcutaneous route. With respect to the pulmonary route, the prostacyclin compounds and compositions) of the present invention may be used in any dosage dispensing device adapted for such administration. The device, in one embodiment, is constructed to ascertain optimum metering accuracy and compatibility of its constructive elements, such as container, valve and actuator with the formulation and could be based on a mechanical pump system, e.g., that of a metered-dose nebulizer, dry powder inhaler, soft mist inhaler, or a nebulizer. For example, pulmonary delivery devices include a jet nebulizer, electronic nebulizer, a soft mist inhaler, and a capsule-based dry powder inhaler.

The prostacyclin or analog thereof, in one embodiment, is sustainly delivered to the lungs, and the prostacyclin or analog thereof is released following administration over a period of time up to about 8 hours, or up to about 12 hours, or up to about 16 hours, or up to about 20 hours, or up to about 24 hours, or up to about 36 hours or up to about 48 hours. In another embodiment, the prostacyclin or analog thereof is sustainly delivered to the lungs, and the prostacyclin or analog thereof is released following administration over a period of time ranging from about 20 hours to about 48 hours, or about 24 hours to about 36 hours or about 30 hours to about 48 hours.

In one embodiment, the pharmaceutical composition is administered in a once-a-day dosing or a twice-a-day dosing regimen to a patient in need thereof. In a further embodiment, the composition is administered via nebulization. In even a further embodiment, the prostacyclin is treprostinil.

In accordance with the present invention, besides via inhalation, administration may be conducted orally, parenterally, subcutaneously, intravenously, or by infusion. The compositions can also be formulated for administration via the nasal passages. Compositions suitable for nasal administration, wherein the carrier is a solid, include a coarse powder having a particle size, for example, in the range of about 3 to about 500 microns which is administered in the manner in which snuff is taken, e.g., by rapid inhalation through the nasal passage from a container of the powder held close up to the nose. Suitable compositions wherein the carrier is a liquid for administration as, for example, nasal spray, nasal drops, or by aerosol administration by nebulizer, include aqueous or oily solutions of the active ingredient.

In another aspect of the invention, a method of treating a disease, disorder or condition other than PH, PAH or PPH is provided. The method comprises administering a therapeutically effective amount of one of the prostacyclin compositions provided herein, for example, a nanoparticle composition comprising a prostacyclin (e.g., treprostinil) or analog thereof, a cationic compound, a surfactant (e.g., PEGylated lipid) and a hydrophobic additive (e.g., squalane) to a patient in need thereof. The diseases, disorders, and conditions include, but are not limited to, chronic thromboembolic pulmonary hypertension, congestive heart failure, peripheral vascular disease, asthma, severe intermittent claudication, immunosuppression, proliferative diseases, cancer such as lung, liver, brain, pancreatic, kidney, prostate, breast, colon, and head-neck cancer, ischemic lesions, neuropathic foot ulcers, and pulmonary fibrosis, kidney function, and interstitial lung disease. In some embodiments, the pharmaceutical formulation comprises one or more additional active ingredients in addition to treprostinil.

U.S. Pat. No. 5,153,222, incorporated by reference herein in its entirety, describes use of treprostinil for treatment of pulmonary hypertension. Treprostinil is approved for the intravenous as well as subcutaneous route, the latter avoiding potential septic events associated with continuous intravenous catheters. U.S. Pat. Nos. 6,521,212 and 6,756,033, each incorporated by reference herein in their entireties, describe administration of treprostinil by inhalation for treatment of pulmonary hypertension, peripheral vascular disease and other diseases and conditions. U.S. Pat. No. 6,803,386, incorporated by reference herein in its entirety, discloses administration of treprostinil for treating cancer such lung, liver, brain, pancreatic, kidney, prostate, breast, colon and head-neck cancer. U.S. Patent Application Publication No. 2005/0165111, incorporated by reference herein in its entirety, discloses treprostinil treatment of ischemic lesions. U.S. Pat. No. 7,199,157, incorporated by reference herein in its entirety, discloses that treprostinil treatment improves kidney functions. U.S. Pat. No. 7,879,909, incorporated by reference herein in its entirety, discloses treprostinil treatment of neuropathic foot ulcers. U.S. Patent Application Publication No. 2008/0280986, incorporated by reference herein in its entirety, discloses treprostinil treatment of pulmonary fibrosis, interstitial lung disease with treprostinil and asthma. U.S. Pat. No. 6,054,486, incorporated by reference herein in its entirety, discloses treatment of peripheral vascular disease with treprostinil. U.S. patent application publication no. 2009/0036465, incorporated by reference herein in its entirety, discloses combination therapies comprising treprostinil. U.S. Patent Application Publication No. 2008/0200449 discloses delivery of treprostinil using a metered dose inhaler. U.S. Pat. Nos. 7,417,070, 7,384,978 and 7,544,713 as well as U.S. Patent Application Publication Nos. 2007/0078095, 2005/0282901, and 2008/0249167, each incorporated by reference herein in their entireties, describe oral formulations of treprostinil and other prostacyclin analogs as well as their use for treatment of a variety of conditions. U.S. Patent Application Publication No. 2012/0004307, incorporated by reference herein, discloses the use of orally administered treprostinil for treatment of Raynaud's phenomenon, systemic sclerosis and digital ischemic lesions.

Additionally, the following references are incorporated by reference for all purposes for practicing the embodiments of the present invention: J. Org. Chem. 2004, 69, 1890-1902, Drug of the Future, 2001, 26(4), 364-374, U.S. Pat. Nos. 5,153,222, 6,054,486, 6,521,212, 6,756,033, 6,803,386, and 7,199,157, U.S. Patent Application Publication Nos. 2005/0165111, 2005/0282903, 2008/0200449, 2008/0280986, 2009/0036465 and 2012/0010159.

In one embodiment, a method is provided for treating a patient in need thereof for congestive heart failure, peripheral vascular disease, asthma, severe intermittent claudication, immunosuppression, proliferative diseases, e.g., cancer such as lung, liver, brain, pancreatic, kidney, prostate, breast, colon and head-neck cancer, ischemic lesions, neuropathic foot ulcers, and pulmonary fibrosis, kidney function and/or interstitial lung disease. In one embodiment, the method comprises administering an effective amount of one of the prostacyclin compositions provided herein, for example, a nanoparticle composition comprising a prostacyclin (e.g., treprostinil) or analog thereof, a cationic compound, a surfactant (e.g., PEGylated lipid) and a hydrophobic additive (e.g., squalane) to the patient. Administration, in one embodiment, is via inhalation (e.g., with a nebulizer or metered dose inhaler), subcutaneous or intravenous. In some embodiments, the pharmaceutical formulation may comprise one or more active ingredients in addition to treprostinil monohydrate.

In one embodiment, a method is provided for treating and/or preventing interstitial lung disease (e.g., pulmonary fibrosis) or asthma, or a condition associated with interstitial lung disease or asthma in a patient in need of such treatment. In a further embodiment, the method comprises administering to the patient an effective amount of one of the prostacyclin compositions provided herein, for example, a nanoparticle composition comprising a prostacyclin (e.g., treprostinil) or analog thereof, a cationic compound, a surfactant (e.g., PEGylated lipid) and a hydrophobic additive (e.g., squalane). The composition or compound, in one embodiment, is delivered via a MDI by the use of a propellant, for example, a chloro-fluorocarbon (CFC) or a fluorocarbon. The patient, in one embodiment, is administered the prostacyclin compound or composition of the invention once daily, twice daily or three times daily. In one embodiment, the administration is with food. In one embodiment, each administration comprises 1 to 5 doses (puffs) from an MDI, for example 1 dose (1 puff), 2 dose (2 puffs), 3 doses (3 puffs), 4 doses (4 puffs) or 5 doses (5 puffs). The MDI, in one embodiment, is small and transportable by the patient. In another embodiment, administration is subcutaneous or intravenous. In another embodiment, intravenous, subcutaneous or inhalation administration of the effective amount of the prostacyclin compound or composition of the invention, for the treatment of interstitial lung disease (e.g., pulmonary fibrosis) or asthma, or a condition associated with interstitial lung disease or asthma, compared to subcutaneous, intravenous or inhalation administration of treprostinil, results in a reduced severity of a systemic adverse events, or a decreased occurrence of a systemic adverse event.

In one embodiment, a method for treating an ischemic disease or condition, such as scleroderma, including systemic sclerosis, or Raynaud's Phenomenon in a patient in need of such treatment is provided. In a further embodiment, the method comprises administering an effective amount of one of the prostacyclin compositions provided herein, for example, a nanoparticle composition comprising a prostacyclin (e.g., treprostinil) or analog thereof, a cationic compound, a surfactant (e.g., PEGylated lipid) and a hydrophobic additive (e.g., squalane), to the patient. Administration, in one embodiment, is via inhalation (e.g., with a nebulizer or metered dose inhaler), subcutaneous or intravenous. In another embodiment, intravenous, subcutaneous or inhalation administration of an effective amount of the prostacyclin compound or composition of the invention, for the treatment of ischemic disease or condition, such as scleroderma, including systemic sclerosis, or Raynaud's Phenomenon, compared to subcutaneous, intravenous or inhalation administration of treprostinil, results in a reduced severity of a systemic adverse events, or a decreased occurrence of a systemic adverse event.

The prostacyclin compositions provided herein, for example, a nanoparticle composition comprising a prostacyclin (e.g., treprostinil) or analog thereof, a cationic compound, a surfactant (e.g., PEGylated lipid) and a hydrophobic additive (e.g., squalane), in one embodiment, are used for treating a patient for a digital ischemic lesion, such as a digital ulcer or a necrotic lesion, or for ameliorating or reducing the number of symptoms and/or functional deficit(s) associated with a digital ischemic lesion. The term “digital ischemic lesion” refers to a lesion on a digit, i.e., a toe or a finger, of a subject, such as a human being. In one embodiment, the digital ischemic lesion may be caused by or associated with an ischemic disease or condition, such as scleroderma, including systemic sclerosis, or Raynaud's Phenomenon. The symptom that may be ameliorated and/or reduced may be, for example, a pain associated with a digital ischemic ulcer and/or scleroderma. In some embodiments, administering a prostacyclin compound or composition provided herein, upon administration to a patient in need of treatment, provides amelioration or reduction of one or more functional deficits associated with a digital ischemic lesion. For example, in one embodiment, the prostacyclin composition provided herein ameliorates or reduces a hand function deficit, i.e., provides an improvement in the hand function of the treated patient. Administration, in one embodiment, is via inhalation (e.g., with a nebulizer or metered dose inhaler), subcutaneous or intravenous. In another embodiment, intravenous, subcutaneous or inhalation administration of an effective amount of the prostacyclin compound or composition of the invention, for the treatment of digital ischemic lesions, compared to subcutaneous, intravenous or inhalation administration of treprostinil, results in a reduced severity of a systemic adverse events, or a decreased occurrence of a systemic adverse event.

In one embodiment, a method for improving kidney function or treating symptoms associated with kidney malfunction or failure in a patient in need thereof is provided. In a further embodiment, the method comprises administering to a subject in need thereof an effective amount of one of the prostacyclin compositions provided herein, for example, a nanoparticle composition comprising a prostacyclin (e.g., treprostinil) or analog thereof, a cationic compound, a surfactant (e.g., PEGylated lipid) and a hydrophobic additive (e.g., squalane). Specific symptoms associated with reduced kidney functions include, for example, abnormally low urination, increased blood levels of creatinine and urea nitrogen, protein leakage in urine and/or pain. Administration, in one embodiment, is via inhalation (e.g., with a nebulizer or metered dose inhaler), subcutaneous or intravenous. In another embodiment, intravenous, subcutaneous or inhalation administration of an effective amount of the prostacyclin compound or composition of the invention, for improvement of kidney functions or amelioration of symptoms associated with kidney malfunction or failure, compared to subcutaneous, intravenous or inhalation administration of treprostinil, results in a reduced severity of a systemic adverse events, or a decreased occurrence of a systemic adverse event.

In one embodiment, a method of treating a cardiovascular disease including congestive heart failure comprises is provided. The method, in one embodiment, comprises administering to a patient in need thereof, a prostacyclin composition provided herein, for example, a nanoparticle composition comprising a prostacyclin (e.g., treprostinil) or analog thereof, a cationic compound, a surfactant (e.g., PEGylated lipid) and a hydrophobic additive (e.g., squalane). Administration, in one embodiment, is via inhalation (e.g., with a nebulizer or metered dose inhaler), subcutaneous or intravenous.

In one embodiment, a method for treating a peripheral vascular disease, including peripheral arterial occlusive disease and intermittent claudication is provided. In one embodiment, the method comprises administering to a patient in need thereof a prostacyclin composition described herein, for example, a nanoparticle composition comprising a prostacyclin (e.g., treprostinil) or analog thereof, a cationic compound, a surfactant (e.g., PEGylated lipid) and a hydrophobic additive (e.g., squalane). In addition to the prostacyclin compounds and compositions provided herein, other pharmacologically active substances may be present in the formulations of the present invention which are known to be useful for treating peripheral vascular disease. For example, the compounds of the invention may be present in combination with trental, a substance known to increase red blood cell deformability. Administration, in one embodiment, is via inhalation (e.g., with a nebulizer or metered dose inhaler), subcutaneous or intravenous.

In one embodiment, a method for treating and/or preventing neuropathic diabetic foot ulcer is provided. In one embodiment, the method comprises administering to a patient in need thereof, a prostacyclin composition described herein, for example, a nanoparticle composition comprising a prostacyclin (e.g., treprostinil) or analog thereof, a cationic compound, a surfactant (e.g., PEGylated lipid) and a hydrophobic additive (e.g., squalane). Administration, in one embodiment, is via inhalation (e.g., with a nebulizer or metered dose inhaler), subcutaneous or intravenous. In addition to the prostacyclin compounds and compositions provided herein, other pharmacologically active substances may be present in the formulations of the present invention which are known to be useful for treating and/or preventing foot ulcers in patients with diabetic neuropathy. For example, the compositions of the invention may be present in combination with analgesics to treat pain, dressing changes, vasodilator medications, and topical or oral antibiotics.

In one embodiment, pulmonary, intravenous or subcutaneous administration of an effective amount of a prostacyclin composition of the present invention for the treatment methods described herein results in a decreased number of side effects, or a reduced severity of one or more side effects (also referred to herein as “adverse events”), compared to the administration of an effective amount of treprostinil, when an effective amount of treprostinil is administered by subcutaneously, intravenously or by inhalation. For example, in one embodiment, a patient in need of treatment with one of the prostacyclin compositions provided herein experiences a reduced severity and/or frequency in cough or a reduced cough response when administered a prostacyclin composition of the invention via inhalation (e.g., nebulization, dry powder inhaler, or via a metered dose inhaler), compared to the severity and/or frequency of cough or cough response elicited by inhalation administration of treprostinil to the patient.

In another embodiment, the prostacyclin composition administered to a patient in need thereof via a pulmonary route by the treatment methods described herein provides a greater pulmonary elimination half-life (t_{1/2}) of the prostacyclin present in the composition, compared to the pulmonary elimination half-life (t_{1/2}) of the prostacyclin, when the unformulated prostacyclin is administered via a pulmonary route (e.g., by nebulization, dry powder inhaler, or a metered dose inhaler) to the patient in need of prostacyclin treatment.

In another embodiment, the prostacyclin composition administered to a patient in need thereof, via the treatment methods described herein provides a greater systemic half-life (t_{1/2}) of the prostacyclin present in the composition, compared to the systemic elimination half-life (t_{1/2}) of the prostacyclin, when the unformulated prostacyclin is administered to the patient. In a further embodiment, administration of the prostacyclin compound and treprostinil comprises either subcutaneous or intravenous administration.

In another embodiment, the prostacyclin composition administered to a patient in need of treatment provides a greater mean pulmonary maximum concentration (Cₘₐₓ) of the prostacyclin present in the composition, or lower plasma Cₘₐₓ of the prostacyclin present in the composition, compared to the pulmonary or plasma Cₘₐₓ of the prostacyclin, when the unformulated prostacyclin (i.e., the free prostacyclin) is administered to the patient. In a further embodiment, administration of the prostacyclin comprises intravenous administration.

In another embodiment, the prostacyclin composition administered to a patient in need of treatment provides a greater mean pulmonary area under the curve (AUC₀₋ₜ) of the prostacyclin present in the composition, compared to the mean pulmonary area under the curve (AUC₀₋ₜ) of the prostacyclin, when the unformulated prostacyclin is administered to the patient. In yet another embodiment, the prostacyclin composition administered to a patient in need thereof provides a greater pulmonary or plasma time to peak concentration (tₘₐₓ) of the prostacyclin, compared to the pulmonary or plasma time to peak concentration (tₘₐₓ) of the prostacyclin, when the unformulated prostacyclin (i.e., the free prostacyclin) is administered to the patient.

In one embodiment, a composition provided herein, for example, a prostacyclin composition provided herein, for example, a nanoparticle composition comprising a prostacyclin (e.g., treprostinil) or analog thereof, a cationic compound, a surfactant and a hydrophobic additive (e.g., squalane) is administered in combination with one or more additional active agents. In some embodiments, such one or more additional active agents can be also administered together with a prostacyclin compound or composition provided herein using a metered dose inhaler. In one embodiment, such one or more additional active agents can be administered separately, i.e., prior to, or subsequent to, the prostacyclin composition provided herein. Particular additional active agents that can be administered in combination with the prostacyclin compositions described herein may depend on a particular disease or condition for treatment or prevention of which a prostacyclin is administered. In some cases, the additional active agent can be a cardiovascular agent such as a cox-2 inhibitor, a rho kinase inhibitor, a calcium channel blocker, a phosphodiesterase inhibitor, an endothelial antagonist, or an antiplatelet agent.

As provided above, the prostacyclin compounds and compositions of the present invention can be delivered to a patient in need thereof via pulmonary, intravenous or subcutaneous route. With respect to the pulmonary route, the prostacyclin compounds and compositions) of the present invention may be used in any dosage dispensing device adapted for such administration. The device, in one embodiment, is constructed to ascertain optimum metering accuracy and compatibility of its constructive elements, such as container, valve and actuator with the formulation and could be based on a mechanical pump system, e.g., that of a metered-dose nebulizer, dry powder inhaler, soft mist inhaler, or a nebulizer. For example, pulmonary delivery devices include a jet nebulizer, electronic nebulizer, a soft mist inhaler, and a capsule-based dry powder inhaler, described in detail herein.

Upon nebulization, the nebulized composition (also referred to as “aerosolized composition”) is in the form of aerosolized particles. The aerosolized composition can be characterized by the particle size of the aerosol, for example, by measuring the “mass median aerodynamic diameter” or “fine particle fraction” associated with the aerosolized composition. “Mass median aerodynamic diameter” or “MMAD” is normalized regarding the aerodynamic separation of aqua aerosol droplets and is determined by impactor measurements, e.g., the Anderson Cascade Impactor (ACI) or the Next Generation Impactor (NGI). The gas flow rate, in one embodiment, is 28 Liter per minute for the ACI and 15 liter per minute for the NGI.

“Geometric standard deviation” or “GSD” is a measure of the spread of an aerodynamic particle size distribution. Low GSDs characterize a narrow droplet size distribution (homogeneously sized droplets), which is advantageous for targeting aerosol to the respiratory system. The average droplet size of the nebulized composition provided herein, in one embodiment is less than 5 μm or about 1 μm to about 5 μm, and has a GSD in a range of 1.0 to 2.2, or about 1.0 to about 2.2, or 1.5 to 2.2, or about 1.5 to about 2.2.

In one embodiment, the mass median aerodynamic diameter (MMAD) of the nebulized composition is about 1 μm to about 5 μm, or about 1 m to about 4 μm, or about 1 m to about 3 m or about 1 m to about 2 μm, as measured by the Anderson Cascade Impactor (ACI) or Next Generation Impactor (NGI). In another embodiment, the MMAD of the nebulized composition is about 5 μm or less, about 4 μm or less, about 3 μm or less, about 2 μm or less, or about 1 μm or less, as measured by cascade impaction, for example, by the ACI or NGI.

In one embodiment, the MMAD of the aerosol of the pharmaceutical composition is less than about 4.9 μm, less than about 4.5 μm, less than about 4.3 μm, less than about 4.2 μm, less than about 4.1 μm, less than about 4.0 μm or less than about 3.5 μm, as measured by cascade impaction.

In one embodiment, the MMAD of the aerosol of the pharmaceutical composition is about 1.0 μm to about 5.0 μm, about 2.0 m to about 4.5 μm, about 2.5 μm to about 4.0 μm, about 3.0 m to about 4.0 μm or about 3.5 μm to about 4.5 μm, as measured by cascade impaction (e.g., by the ACI or NGI).

“Fine particle fraction” or “FPF”, as used herein, refers to the fraction of the aerosol having a particle size less than 5 m in diameter, as measured by cascade impaction. FPF is usually expressed as a percentage.

In one embodiment, the FPF of the aerosolized composition is greater than or equal to about 50%, as measured by the ACI or NGI, greater than or equal to about 60%, as measured by the ACI or NGI or greater than or equal to about 70%, as measured by the ACI or NGI. In another embodiment, the FPF of the aerosolized composition is about 50% to about 80%, or about 50% to about 70% or about 50% to about 60%, as measured by the NGI or ACI.

In one embodiment, a dry powder inhaler (DPI) is employed as the inhalation delivery device for the compositions of the present invention. In one embodiment, the DPI generates particles having an MMAD of from about 1 μm to about 10 μm, or about 1 μm to about 9 μm, or about 1 μm to about 8 μm, or about 1 μm to about 7 μm, or about 1 μm to about 6 μm, or about 1 m to about 5 μm, or about 1 μm to about 4 μm, or about 1 μm to about 3 μm, or about 1 μm to about 2 μm in diameter, as measured by the NGI or ACI. In another embodiment, the DPI generates a particles having an MMAD of from about 1 μm to about 10 μm, or about 2 μm to about 10 μm, or about 3 μm to about 10 μm, or about 4 μm to about 10 μm, or about 5 μm to about 10 μm, or about 6 μm to about 10 μm, or about 7 m to about 10 μm, or about 8 μm to about 10 μm, or about 9 μm to about 10 μm, as measured by the NGI or ACI.

In one embodiment, the MMAD of the particles generated by the DPI is about 1 jm or less, about 9 μm or less, about 8 μm or less, about 7 μm or less, 6 μm or less, 5 μm or less, about 4 μm or less, about 3 μm or less, about 2 μm or less, or about 1 μm or less, as measured by the NGI or ACI.

In one embodiment, the MMAD of the particles generated by the DPI is less than about 9.9 μm, less than about 9.5 μm, less than about 9.3 μm, less than about 9.2 μm, less than about 9.1 μm, less than about 9.0 μm, less than about 8.5 μm, less than about 8.3 μm, less than about 8.2 μm, less than about 8.1 μm, less than about 8.0 μm, less than about 7.5 μm, less than about 7.3 μm, less than about 7.2 μm, less than about 7.1 μm, less than about 7.0 μm, less than about 6.5 μm, less than about 6.3 μm, less than about 6.2 μm, less than about 6.1 μm, less than about 6.0 μm, less than about 5.5 μm, less than about 5.3 μm, less than about 5.2 μm, less than about 5.1 μm, less than about 5.0 μm, less than about 4.5 μm, less than about 4.3 μm, less than about 4.2 μm, less than about 4.1 μm, less than about 4.0 μm or less than about 3.5 μm, as measured by the NGI or ACI.

In one embodiment, the MMAD of the particles generated by the DPI is about 1.0 jm to about 10.0 μm, about 2.0 μm to about 9.5 μm, about 2.5 μm to about 9.0 μm, about 3.0 μm to about 9.0 μm, about 3.5 μm to about 8.5 μm or about 4.0 μm to about 8.0 μm.

In one embodiment, the FPF of the prostacyclin particulate composition generated by the DPI is greater than or equal to about 40%, as measured by the ACI or NGI, greater than or equal to about 50%, as measured by the ACI or NGI, greater than or equal to about 60%, as measured by the ACI or NGI, or greater than or equal to about 70%, as measured by the ACI or NGI. In another embodiment, the FPF of the aerosolized composition is about 40% to about 70%, or about 50% to about 70% or about 40% to about 60%, as measured by the NGI or ACI.

Another aspect of the present invention relates to a system for treating or providing prophylaxis against pulmonary hypertension, e.g., pulmonary arterial hypertension, or portopulmonary hypertension. In one embodiment, the system comprises a pharmaceutical composition comprising a prostacyclin or analog thereof, a cationic compound, and a surfactant; and an inhalation device. In one embodiment, the inhalation device is a nebulizer. In a further embodiment, the prostacyclin composition comprises a hydrophobic additive (e.g., squalane) and the composition comprises a plurality of nanoparticles.

A nebulizer type inhalation delivery device can contain the compositions of the present invention as a solution, usually aqueous, or a suspension. For example, the prostacyclin composition can be suspended in saline and loaded into the inhalation delivery device. In generating the nebulized spray of the compositions for inhalation, the nebulizer delivery device may be driven ultrasonically, by compressed air, by other gases, electronically or mechanically (e.g., vibrating mesh or aperture plate). Vibrating mesh nebulizers generate fine particle, low velocity aerosol, and nebulize therapeutic solutions and suspensions at a faster rate than conventional jet or ultrasonic nebulizers. Accordingly, the duration of treatment can be shortened with a vibrating mesh nebulizer, as compared to a jet or ultrasonic nebulizer. Vibrating mesh nebulizers amenable for use with the methods described herein include the Philips Respironics I-Neb®, the Omron MicroAir, the Nektar Aeroneb®, and the Pari eFlow®. The nebulizer, in one embodiment, is a single-use (e.g., disposable) or a multi-use nebulizer. In one embodiment, the system provided herein comprises a nebulizer selected from an electronic mesh nebulizer, pneumonic (jet) nebulizer, ultrasonic nebulizer, breath-enhanced nebulizer and breath-actuated nebulizer. In one embodiment, the nebulizer is portable.

The inhalation delivery device can be a nebulizer, dry powder inhaler, or a metered dose inhaler (MDI), or any other suitable inhalation delivery device known to one of ordinary skill in the art. The device can contain and be used to deliver a single dose of the prostacyclin composition or the device can contain and be used to deliver multi-doses of the composition of the present invention.

A nebulizer type inhalation delivery device can contain the compositions of the present invention as a solution, usually aqueous, or a suspension. For example, the prostacyclin compound or composition can be suspended in saline and loaded into the inhalation delivery device. In generating the nebulized spray of the compositions for inhalation, the nebulizer delivery device may be driven ultrasonically, by compressed air, by other gases, electronically or mechanically (e.g., vibrating mesh or aperture plate). Vibrating mesh nebulizers generate fine particle, low velocity aerosol, and nebulize therapeutic solutions and suspensions at a faster rate than conventional jet or ultrasonic nebulizers. Accordingly, the duration of treatment can be shortened with a vibrating mesh nebulizer, as compared to a jet or ultrasonic nebulizer. Vibrating mesh nebulizers amenable for use with the methods described herein include the Philips Respironics I-Neb®), the Omron MicroAir, the Nektar Aeroneb®, and the Pari eFlow®.

The nebulizer may be portable and hand held in design, and may be equipped with a self contained electrical unit. The nebulizer device may comprise a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer may be employed to ensure the sizing of particles is optimal for positioning of the particle within, for example, the pulmonary membrane.

In another embodiment, the nebulizer described herein generates an aerosol of the prostacyclin pharmaceutical composition at a rate greater than about 0.35 g per minute, greater than about 0.40 g per minute, greater than about 0.50 g per minute, or about 0.60 g per minute to about 0.70 g per minute. In a further embodiment, the Fine Particle Fraction (FPF) of the aerosol is greater than or equal to about 50%, as measured by cascade impaction, greater than or equal to about 60%, as measured by cascade impaction, or greater than or equal to about 70%, as measured by cascade impaction.

The principle of operation of a pneumonic nebulizer is generally known to those of ordinary skill in the art and is described, e.g., in Respiratory Care, Vol. 45. No. 6, pp. 609-622 (2000), incorporated by reference herein for all purposes. Briefly, a pressurized gas supply is used as the driving force for liquid atomization in a pneumatic nebulizer. Compressed gas is delivered, which causes a region of negative pressure. The solution to be aerosolized is then delivered into the gas stream and is sheared into a liquid film. This film is unstable and breaks into droplets because of surface tension forces. Smaller particles, i.e., particles with the MMAD and FPF properties described herein, can then be formed by placing a baffle in the aerosol stream. In one pneumonic nebulizer embodiment, gas and solution is mixed prior to leaving the exit port (nozzle) and interacting with the baffle. In another embodiment, mixing does not take place until the liquid and gas leave the exit port (nozzle). In one embodiment, the gas is air, O₂ and/or CO₂.

In one embodiment, droplet size and output rate can be tailored in a pneumonic nebulizer. However, consideration should be paid to the composition being nebulized, and whether the properties of the composition (e.g., % associated prostacyclin) are altered due to the modification of the nebulizer. For example, in one embodiment, the gas velocity and/or pharmaceutical composition velocity is modified to achieve the output rate and droplet sizes of the present invention. Additionally or alternatively, the flow rate of the gas and/or solution can be tailored to achieve the droplet size and output rate of the invention. For example, an increase in gas velocity, in one embodiment, decreased droplet size. In one embodiment, the ratio of pharmaceutical composition flow to gas flow is tailored to achieve the droplet size and output rate of the invention. In one embodiment, an increase in the ratio of liquid to gas flow increases particle size.

Nebulization time, in one embodiment, is reduced by increasing the flow to power the nebulizer. See, e.g., Clay et. al. (1983). Lancet 2, pp. 592-594 and Hess et al. (1996). Chest 110, pp. 498-505, each of which is incorporated by reference herein for all purposes.

In one embodiment, a reservoir bag or chamber is used to capture aerosol during the nebulization process, and the aerosol is subsequently provided to the subject via inhalation. In another embodiment, the nebulizer provided herein includes a valved open-vent design. In this embodiment, when the patient inhales through the nebulizer, nebulizer output is increased. During the expiratory phase, a one-way valve diverts patient flow away from the nebulizer chamber.

In one embodiment, the nebulizer provided herein is a continuous nebulizer. In other words, refilling the nebulizer with the pharmaceutical composition while administering a dose is not needed.

In one embodiment, a vibrating mesh nebulizer is used to deliver the prostacyclin composition of the invention to a patient in need thereof. In one embodiment, the nebulizer membrane vibrates at an ultrasonic frequency of about 50 kHz to about 500 kHz, about 100 kHz to about 450 kHz, about 150 kHz to about 400 kHz, or about 200 kHz to about 350 kHz.

In one embodiment, the nebulizer provided herein does not use an air compressor and therefore does not generate an air flow. In one embodiment, aerosol is produced by the aerosol head which enters the mixing chamber of the device. When the patient inhales, air enters the mixing chamber via one-way inhalation valves in the back of the mixing chamber and carries the aerosol through the mouthpiece to the patient. On exhalation, the patient's breath flows through the one-way exhalation valve on the mouthpiece of the device. In one embodiment, the nebulizer continues to generate aerosol into the mixing chamber which is then drawn in by the subject on the next breath—and this cycle continues until the nebulizer medication reservoir is empty.

The compositions provided herein, in one embodiment, are used for treatment of PH, PAH or PPH via inhalation (e.g., nebulization). The composition, in one embodiment, is administered via a nebulizer, which provides an aerosol mist of the composition for delivery to the lungs of a patient in need thereof.

In one embodiment, the nebulizer generates an aerosol of the pharmaceutical composition at a rate of about 0.1 to 1.0 mL/min. In one embodiment, the mass median aerodynamic diameter (MMAD) of the nebulized composition is about 1 μm to about 5 μm, or about 1 μm to about 4 μm, or about 1 μm to about 3 μm or about 1 μm to about 2 μm, as measured by the Anderson Cascade Impactor (ACI) or Next Generation Impactor (NGI). In another embodiment, the MMAD of the nebulized composition is about 5 μm or less, about 4 μm or less, about 3 μm or less, about 2 μm or less, or about 1 μm or less, as measured by cascade impaction.

In one embodiment, the system provided herein comprises a prostacyclin composition, for example, a treprostinil composition, e.g., a treprostinil solid nanoparticle formulation.

Another aspect of the present invention relates to a prostacyclin aerosol comprising a particulate composition, which comprises a prostacyclin or analog thereof, a cationic compound and a surfactant. In one embodiment, the particulate composition is a solid lipid nanoparticulate composition. In one embodiment, the aerosol is generated at a rate of about 0.1 to about 1.0 mL/min.

In one embodiment, prior to aerosolization of the prostacyclin composition, about 60% to about 100% of the prostacyclin present in the composition is in particle form. In a further embodiment, the prostacyclin is treprostinil, epoprostenol, or iloprost. In another embodiment, prior to nebulization, about 65% to about 99%, about 75% to about 99%, about 85% to about 99%, about 95% to about 99%, or about 97% to about 99% is in particle form.

In another embodiment, prior to aerosolization of the prostacyclin composition, about 85% to about 99%, or about 90% to about 99% or about 95% to about 99% or about 96% to about 99% of the prostacyclin present in the composition is in particle form. In a further embodiment, the prostacyclin is treprostinil, epoprostenol, or iloprost. In another embodiment, prior to nebulization, about 98% of the prostacyclin present in the composition is in particle form.

In one embodiment, the FPF of the aerosolized composition is greater than or equal to 50%, greater than or equal to 60%, greater than or equal to 70%, greater than or equal to 80%, greater than or equal to 90%, greater than or equal to 95%, greater than or equal to 97.5%, or greater than or equal to 99%, as measured by cascade impaction. In a further embodiment, the composition comprises treprostinil. In a further embodiment, the composition comprises a cationic lipid. In even a further embodiment, the composition is a micellar composition.

In one embodiment, the inhalation device described herein generates an aerosol (i.e., achieves a total output rate) of the prostacyclin pharmaceutical composition at a rate of about 0.1 to 1.0 mL/min. An aerosol of the prostacyclin composition, in one embodiment, is generated at a rate greater than about 0.25 g per minute, greater than about 0.35 g per minute, greater than about 0.45 g per minute, greater than about 0.55 g per minute, greater than about 0.60 g per minute, greater than about 0.65 g per minute or greater than about 0.70 g per minute. In another embodiment, the inhalation device described herein generates an aerosol (i.e., achieves a total output rate) of the prostacyclin pharmaceutical composition at about 0.53 g per minute to about 0.80 g per minute, at about 0.53 g per minute to about 0.70 g per minute, about 0.55 g per min to about 0.70 g per minute, about 0.53 g per minute to about 0.65 g per minute, or about 0.60 g per minute to about 0.70 g per minute. In one embodiment, the inhalation device of the system is a nebulizer or a dry powder inhaler.

Upon nebulization, in one embodiment, the particles in the pharmaceutical composition leak drug. In one embodiment, the amount of particle associated prostacyclin post-nebulization is about 25% to about 90%, or about 40% to about 80% or about 50% to about 70%. These percentages are also referred to herein as “percent associated prostacyclin post-nebulization.” As provided herein, in one embodiment, the composition provided herein comprises a plurality of particles, which comprise a prostacyclin, e.g., treprostinil. In one embodiment, the percent associated prostacyclin post-nebulization is from about 30% to about 80%.

In one embodiment, the percent associated prostacyclin post-nebulization is measured by reclaiming the aerosol from the air by condensation in a cold-trap, and the liquid is subsequently assayed for free and encapsulated prostacyclin (associated prostacyclin).

In one embodiment, the MMAD of the aerosol of the pharmaceutical composition is less than about 4.9 μm, less than about 4.5 μm, less than about 4.3 μm, less than about 4.2 μm, less than about 4.1 μm, less than about 4.0 μm or less than about 3.5 μm, as measured by cascade impaction.

In one embodiment, the MMAD of the aerosol of the pharmaceutical composition is about 1.0 μm to about 5.0 μm, about 2.0 μm to about 4.5 μm, about 2.5 μm to about 4.0 μm, about 3.0 μm to about 4.0 μm or about 3.5 μm to about 4.5 μm, as measured by cascade impaction.

### EXAMPLES

The present invention is further illustrated by reference to the following examples However, it should be noted that these Examples, like the embodiments described above, are illustrative and are not to be construed as restricting the scope of the invention in any way.

Treprostinil compositions used in these experiments may include treprostinil either in the form of a free acid or a salt (FIG. 1). Treprostinil can be synthesized, for example, by the methods disclosed in U.S. Pat. Nos. 6,765,117 and 8,497,393. Syntheses of prostaglandin derivatives are described in U.S. Pat. No. 4,668,814. The disclosures of U.S. Pat. Nos. 6,765,117; 8,497,393; and 4,668,814 are each incorporated by reference in their entireties for all purposes.

The following assays were used in the examples provided below.

### Filtration Assay

To measure the percentage of free treprostinil (i.e., unassociated), 500 μL of treprostinil nanoparticle formulations at concentrations of 1 mM, 100 μM, and 10 μM were used. Samples were loaded onto Vivacon spin filters with 30000 Da molecular weight cut off (MWCO) and centrifuged for 25 min. at 5000×g. The filtrate was collected and its treprostinil content was measured by HPLC. Treprostinil content in filtrate is equivalent to ‘free treprostinil,’ i.e., non nanoparticle-associated treprostinil, and expressed as percentage of total treprostinil content pre filtration.

### Particle Size Assay

To measure particle size of the treprostinil compositions, 50 μL of sample diluted in 950 μL of deionized H₂O (filtered through 0.02 μm filter) was aliquoted into a disposable plastic cuvette and analyzed on a Mobius particle analyzer (Wyatt, Calif.). Data were collected and averaged for 10 acquisitions, 3 seconds per acquisition at 23° C.

### HPLC Assay

Treprostinil concentration was measured by HPLC analysis using the Waters Alliance 2695 system with a Corona detector and PDA detector. UV absorbance was measured at 270 nm. Column ACE 3 C8 4.6×50 (Mac-Mod Analytical) was used.

Mobile phase A contained 25% acetonitrile 25% methanol, 50% water, 0.1% formic acid, and 0.01%, triethylamine. Mobile phase B contained 50% acetonitrile, 50% methanol, 0.1% formic acid, and 0.01%, triethylamine. Mobile phase gradient was used with phase B increasing from 40 to 95% over 5 min.

### Example 1

### Synthesis of Treprostinil Compositions

Treprostinil compositions of the present invention were prepared as follows. A mixture of treprostinil, cationic lipid, hydrophobic filler, and a PEGylated lipid at a desired molar ratio were dissolved in ethanol. Table 2 shows a representative number of treprostinil compositions made by the method. Additionally, the average particle size (nm) for each composition is provided at the last column.

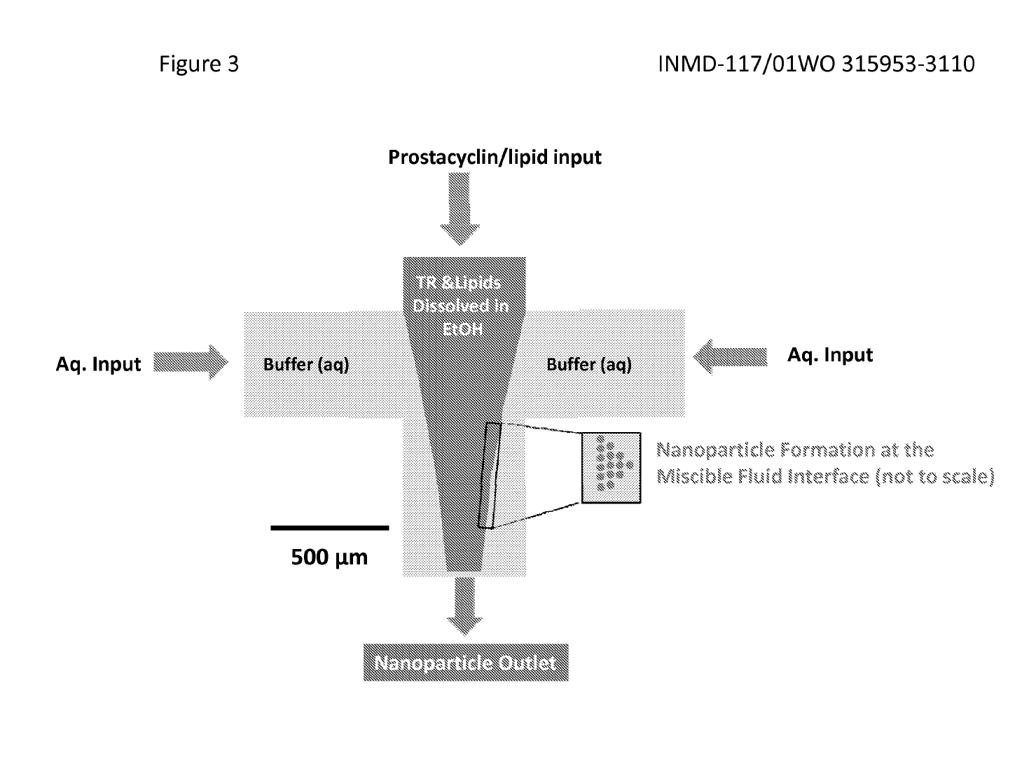

Total concentration of components in ethanol solution was usually 40 mM or 80 mM. Certain volumes of the solution (usually 1 mL) were mixed in-line with 9 part of an aqueous buffer by combining two streams in a mixing cross with a total flow rate of 100 mL/min. The flow rate ratio of buffer (aqueous input) to lipid was approximately 20:1. See FIG. 3 for a schematic of the mixing process.

TABLE 2
Representative treprostinil compositions made by the methods of Example 1.

| | Cat | Hyd | | TRP | TRP | TRP | TRP | |
|---|---|---|---|---|---|---|---|---|
| | Lipid (CL) | Additive | PEG-lipid | (mol | total | Free1 | Free2 | Size |
| Composition | (mol %) | (mol %) | (mol %) | %) | mM | (%) | (%) | (nm) |
| T527** | diC18dMA | Squalane | Chol-PEG2K | 5 | 0.18 | 8 | 23 | 59 |
| | (15%) | (60%) | (10%) | | | | | |
| T550** | diC18dMA | Squalane | Chol-PEG2K | 2 | 0.08 | 3 | 15 | 45 |
| | (10%) | (68%) | (10%) | | | | | |
| T490 | diC18dMA | Chol | Chol-PEG2K | 15 | 0.59 | 30 | 72 | 70 |
| | (30%) | (25%) | (30%) | | | | | |
| T489 | diC18dMA | Chol | Chol-PEG2K | 15 | 0.58 | 28 | 66 | 65 |
| | (30%) | (30%) | (25%) | | | | | |
| T482 | diC18dMA | Chol | Chol-PEG2K | 15 | 0.65 | 46 | 78 | 49 |
| | (30%) | (35%) | (20%) | | | | | |
| T465 | diC18dMA | SqualEne | DSG-PEG2K | 5 | 0.20 | 1.7 | 13 | 22 |
| | (15%) | (60%) | (20%) | | | | | |
| T464 | diC18dMA | SqualEne | DSG-PEG2K | 15 | 0.58 | 8.1 | 15 | 18 |
| | (45%) | (15%) | (25%) | | | | | |
| T465 | diC18dMA | SqualEne | DSG-PEG2K | 5 | 0.20 | 5.3 | 21 | 32 |
| | (15%) | (60%) | (20%) | | | | | |
| T460 | diC18dMA | SqualEne | DSG-PEG2K | 10 | 0.37 | 2.0 | 19 | 23 |
| | (30%) | (40%) | (20%) | | | | | |
| T459 | diC18dMA | SqualEne | DSG-PEG2K | 10 | 0.39 | 4.9 | 20 | 25 |
| | (30%) | (45%) | (15%) | | | | | |
| T452 | diC18dMA | SqualEne | DSG-PEG2K | 5 | 0.34 | 13 | 24 | 16 |
| | (15%) | (60%) | (20%) | | | | | |
| T448 | diC18dMA | triCl2 | DSG-PEG2K | 1.5 | 1.30 | 15 | 41 | 36 |
| | (15%) | (40%) | (15%) | | | | | |
| T449 | diC18dMA | triCl2 | DSG-PEG2K | 15 | 1.00 | 18 | 49 | 36 |
| | (30%) | (45%) | (10%) | | | | | |
| T450 | diC18dMA | triCl2 | DSG-PEG2K | 15 | 1.10 | 56 | 77 | 87 |
| | (30%) | (50%) | (5%) | | | | | |
| T447 | diC18dMA | triCl2 | DSG-PEG2K | 15 | 1.10 | 18 | 40 | 27 |
| | (30%) | (35%) | (20%) | | | | | |
| T440 | diC18dMA | Squalane | DSG-PEG2K | 5 | 0.40 | 3.6 | 13 | 68 |
| | (10%) | (65%) | (20%) | | | | | |
| T441 | diC18dMA | Squalane | DSG-PEG2K | 5 | 0.40 | 0.9 | 11 | 61 |
| | (15%) | (60%) | (20%) | | | | | |
| T420 | diC18dMA | Squalane | DSG-PEG2K | 15 | 1.20 | 11 | 23 | |
| | (30%) | (35%) | (20%) | | | | | |
| T427 | diC18dMA | Squalane | DSG-PEG2K | 10 | 0.80 | 8.7 | 17 | 71 |
| | (20%) | (50%) | (20%) | | | | | |
| T428 | diC18dMA | Squalane | DSG-PEG2K | 3 | 0.30 | 2.4 | 27 | 97 |
| | (7%) | (70%) | (20%) | | | | | |
| T429 | diC18dMA | Squalane | DSG-PEG2K | 17 | 1.40 | 39 | 47 | 36 |
| | (35%) | (25%) | (23%) | | | | | |
| T430 | diC18dMA | Squalane | DSG-PEG2K | 12 | 0.90 | 21 | 25 | 66 |
| | (23%) | (50%) | (15%) | | | | | |
| T431 | diC18dMA | Squalane | DSG-PEG2K | 7 | 0.60 | 5.0 | 12 | 100 |
| | (14%) | (70%) | (9%) | | | | | |
| T416 | — | Squalane | DSG-PEG2K | 15 | 1.2 | NM | NM | NM |
| | | (20%) | (65%) | | | | | |

**T527 and T550 each include 10% mol DOPC (dioleoyl phosphatidylcholine).
TRP = treprostinil;
triCl2 = tridodecanoylglycerol;
diC18dMA = dioctadecyldimethyl ammonium bromide;
Chol-PEG2K = Cholesterol-PEG2000;
DSG-PEG2K = disteraroylglycerol-PEG2000.
TRP Free1 (as % of total TRP) is measured at total TRP concentration 100 μM.
TRP Free2 (as % of total TRP) is measured at 10 μM.
NM = not measured.

A Gilson 402 syringe pump was used to deliver the ethanol solution. A peristaltic pump was used to deliver the aqueous buffer solution. After mixing, the treprostinil nanoparticles spontaneously formed. Ethanol solvent remaining in the final mixture was then removed by blowing a stream of nitrogen gas, or sparging nitrogen gas.

As shown in Table 3, compositions comprising different types of cationic lipids were made. Of the different types of cationic lipids, trioctyl-amine (triC8-amine) produced formulations with least treprostinil retention (highest free %). Compositions comprising didodecyldimethyl ammonium, as bromide salt (diC12dMA), exhibited a high treprostinil retention. (Table 3).

TABLE 3

| | | | TRP/CL/ | | | | |
|---|---|---|---|---|---|---|---|
| | | PEG-lipid | PEG-lipid | | TRP total | Ethanol | TRP free |
| Batch | Cationic lipid | (surfactant) | mol ratio | Buffer | (mM) | (%) | (%) |
| 1 | C16-amine | PE-PEG3K | 1:1:2 | Citrate 20 | 1.2 | 8.4 | 43.8 |
| | | | | mM | | | |
| 2 | C16-amine | DSPE- | 1:2:1 | Citrate 20 | 0.8 | 8.8 | 13.2 |
| | | PEG3K | | mM | | | |
| 3 | C16-amine | DSPE- | 1:2:1 | BES20, | 0.8 | 8.8 | 13.8 |
| | | PEG3K | | NaCl100 | | | |
| 4 | DPTAP | DSPE- | 1:2:1 | BES20 | 1.0 | 7.3 | 11.2 |
| | | PEG3K | | | | | |
| 5 | diC12dMA | DSPE- | 1:2:1 | Citrate 20 | 1.0 | 7.5 | 8.7 |
| | | PEG3K | | mM | | | |
| 6 | triC8- | DSPE- | 1:2:1 | Citrate 20 | 1.0 | 7.5 | 56.7 |
| | ammonium | PEG3K | | mM | | | |
| 7 | triC8- | DSPE- | 1:1:1 | Citrate 20 | 1.0 | 7.5 | 73.9 |
| | ammonium | PEG3K | | mM | | | |
| 8 | triC8- | DSPE- | 2:2:1 | Citrate 20 | 1.0 | 7.5 | 83.3 |
| | ammonium | PEG3K | | mM | | | |
| 9 | diC12dMA | DSPE- | 2:3:1 | BES20, | 1.0 | 7.5 | 17.7 |
| | | PEG3K | | NaCl50 | | | |
| 10 | diC12dMA | DSPE- | 4:5:1 | BES20, | 1.0 | 7.5 | 17.3 |
| | | PEG3K | | NaCl50 | | | |
| 11 | diC12dMA | DSPE- | 2:3:1 | Citrate 20 | 2.0 | 3.9 | 11.9 |
| | | PEG3K | | mM | | | |
| 12 | diC12dMA | DSPE- | 2:3:1 | Citrate 20 | 2.0 | 3.9 | 13.8 |
| | | PEG3K | | NaCl50 | | | |
| 13 | diC12dMA | DSPE- | 2:3:1 | Citrate 20 | 2.0 | 3.9 | 14.9 |
| | | PEG3K | | NaCl100 | | | |

C16-amine = hexadecylamine;
triC8-ammonium = trioctyl-ammonium;
DPTAP = 1,2-dipalmitoyl-3-trimethylammonium-propane;
diC12dMA = didodecyldimethyl ammonium bromide;
PE-PEG3K = phosphatidylethanolamine-PEG3000;
DSPE-PEG3K = distearoylphosphatidylethanolamine-PEG3000.

### Example 2

### Particle Size Characterization of Treprostinil Compositions

All particle size measurements were performed using a Wyatt Technology Mobius™ Zeta Potential/Particle Sizing Instrument in Quasi-elastic light scattering (QELS) mode. Composition aliquots were diluted 10-fold in pre-filtered (0.02 μm pore filter) ultrapure of deionized H₂O. Light scattering data were collected and converted into particle size and size distribution using Dynamics® v. 7.2.4 instrument software. Reported average particle size diameter was based on the cumulants model, which mathematically fits particle diffusion constants (determined by the raw scattering intensities of particles in a suspension) to obtain the particle size mean and a distribution of particle sizes around the mean diameter. The testing samples included T426, T420, T427, and T428. (Table 4).

TABLE 4

| | | Hydrophobic | | Cationic |
|---|---|---|---|---|
| | TRP | additive | PEG-lipid | compound |
| Composition | (mol %) | (mol %) | (mol %) | (mol %) |
| T420 | 15% | Squalane | DSG-PEG2K | diC18dMA |
| | | (35%) | (20%) | (30%) |
| T426 | 18.3% | Squalane | DSG-PEG2K | diC18dMA |
| | | (25%) | (20%) | (36.7%) |
| T427 | 10% | Squalane | DSG-PEG2K | diC18dMA |
| | | (50%) | (20%) | (20%) |
| T428 | 3.3% | Squalane | DSG-PEG2K | diC18dMA |
| | | (70%) | (20%) | (6.7%) |
| T429 | 17.3% | Squalane | DSG-PEG2K | diC18dMA |
| | | (25%) | (23.1%) | (34.6%) |
| T430 | 11.5% | Squalane | DSG-PEG2K | diC18dMA |
| | | (50%) | (15.4%) | (23.1%) |
| T431 | 6.9% | Squalane | DSG-PEG2K | diC18dMA |
| | | (70%) | (9.3%) | (13.8%) |

TRP = treprostinil;
DSG-PEG2K = disteraroylglycerol-PEG2000;
diC18dMA = dioctadecyldimethyl ammonium bromide.

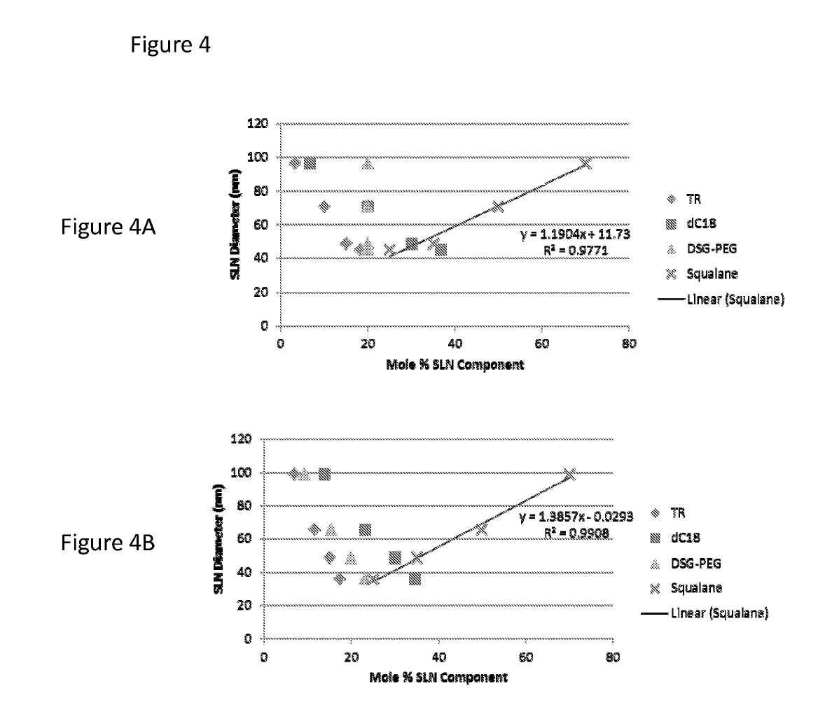

It was found from these experimental results that the particle size (average particle diameter) of treprostinil compositions increases with increased Squalane (hydrophobic filler). As shown in FIG. 4, squalane content is inversely related to the other components for particle size. In FIG. 4A, the treprostinil and cationic lipid ratio was fixed, and SDG-PEG was present at 20% for all samples (T426, T420, T427 and T428). FIG. 4B (T429, T420, T430, T431) shows the nanoparticle diameter of treprostinil compositions having a fixed treprostinil/cationic lipid/PEG ratio. It was observed that the effect of squalane was slightly more pronounced in the sample of FIG. 4B where the TR/CL/PEG ratio is fixed (e.g. PEG % is reduced with other lipid components) as determined by the slope of the fit line. These data suggest that to some degree, PEG percentage also plays a role in particle size for treprostinil compositions.

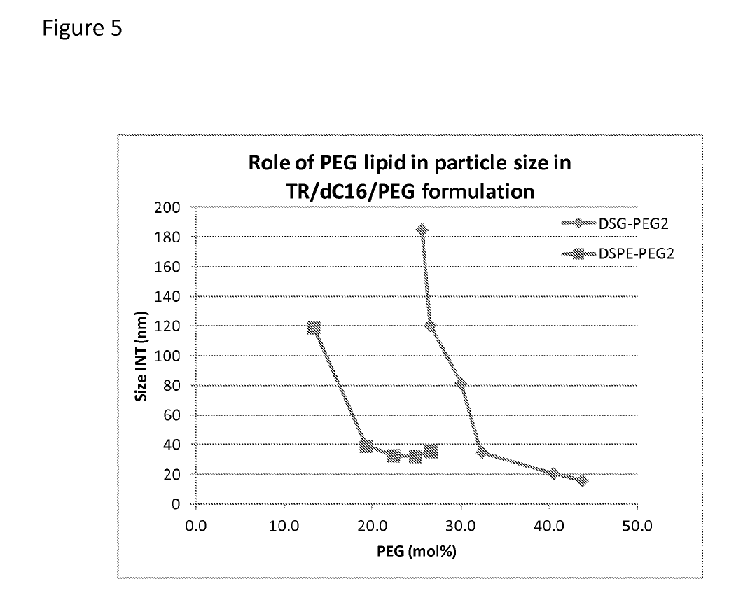

FIG. 5 shows the role of PEGylated-lipid concentration in particle size of compositions comprising treprostinil, dC16 (cationic lipid) and either DSG-PEG2000 or DSPE-PEG2K. As shown in FIG. 5. PEGylated lipid concentration is inversely correlated to particle size, i.e., the size of the particles decreases with increasing mol % of PEGyated lipid for both compositions. The particle size of the compositions comprising DSPE-PEG2K plateaus around 20% PEG (mol %).

### Example 3

### Treprostinil Nanoparticle Association as a Function of Composition Components

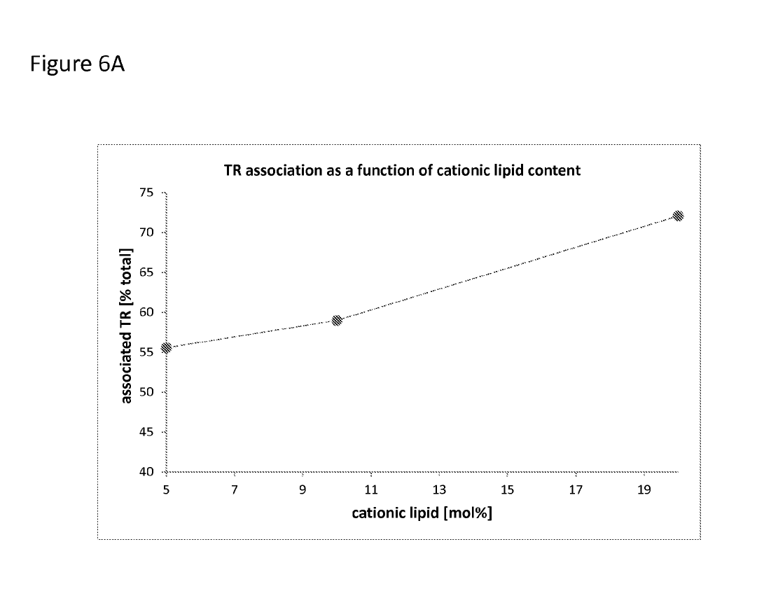

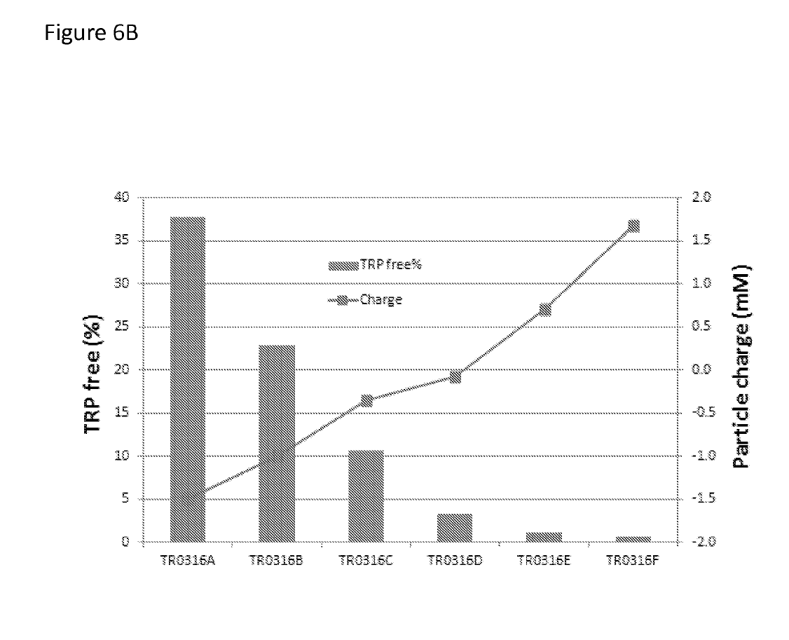

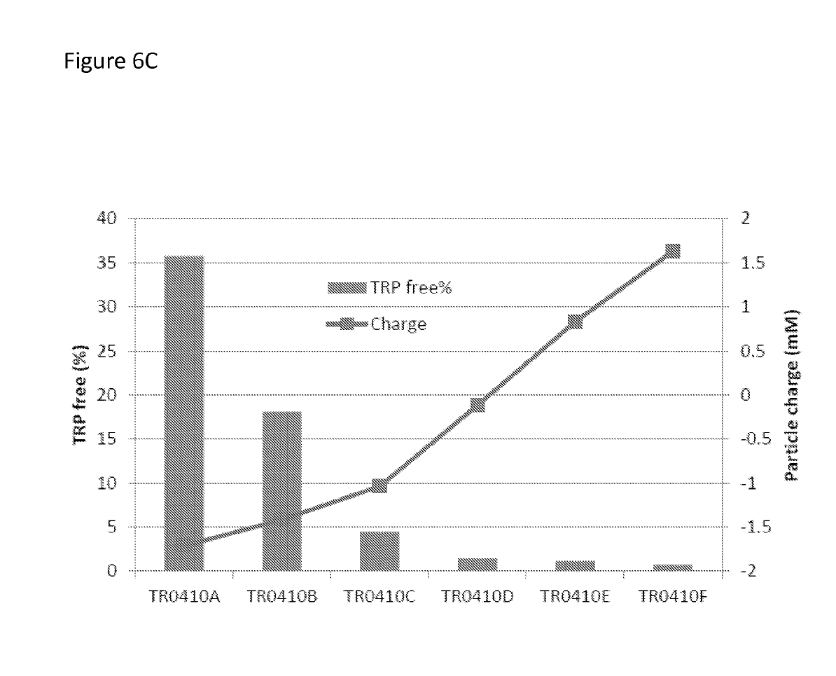

The effect of various composition components on the degree of treprostinil association was measured. The compositions used in the study were T590, T591 and T592 (FIG. 6A), the components of which are provided in Table 5, below. Treprostinil free % was measured as described, after diluting composition to a total treprostinil concentration of 10 μM. Treprostinil associated was calculated as 100%—TRPfree %. It was found that treprostinil association increased with increasing cationic lipid content (FIG. 6A).

TABLE 5

| | | Hydrophobic | | Cationic |
|---|---|---|---|---|
| | TR | additive | PEG-lipid | compound |
| Composition | (mol %) | (mol %) | (mol %) | (mol %) |
| T590 | 5% | Squalane | Chol-PEG2K | diC18dMA |
| | | (70%) | (20%) | (5%) |
| T591 | 5% | Squalane | Chol-PEG2K | diC18dMA |
| | | (65%) | (20%) | (10%) |
| T592 | 5% | Squalane | Chol-PEG2K | diC18dMA |
| | | (55%) | (20%) | (20%) |

Chol-PEG2K = cholesterol-PEG2000.
diC18dMA = dioctadecyldimethyl ammonium bromide.

TABLE 6

| | Cationic | (CL)/TRP | | CL/TRP mo- |
|---|---|---|---|---|
| | Lipid | molar ratio | | lar ratio in |
| Composition | (CL) | total | PEG-lipid | nonoparticle |
| TR0316A | diC12dMA | 0.76 | DSPE-PEG3K | 1.21 |
| TR0316B | diC12dMA | 1.03 | DSPE-PEG3K | 1.34 |
| TR0316C | diC12dMA | 1.31 | DSPE-PEG3K | 1.46 |
| TR0316D | diC12dMA | 1.45 | DSPE-PEG3K | 1.50 |
| TR0316E | diC12dMA | 1.66 | DSPE-PEG3K | 1.68 |
| TR0316F | diC12dMA | 1.90 | DSPE-PEG3K | 1.92 |

diC12dMA = didodecyldimethyl-ammonium bromide.
DSPE-PEG3K = distearoylphosphatidylethanolamine-PEG3000.
CL/TRP mole ratio in nanoparticle was calculated by subtracting the measured TRP free and was used to determine the particle charge.

FIG. 6B also shows the measured amount of free treprostinil (%), which is inverse to the associated treprostinil, as a function of cationic lipid content. Table 6 provides the cationic lipid/treprostinil molar ratio for each composition tested. PEGylated lipid:treprostinil molar ratio in these compositions was kept at a constant ratio of 0.5. Consistent with FIG. 6A, the amount of associated treprostinil correlates with increasing cationic lipid content (FIG. 6B). FIG. 6B also shows the total charge of the particles for each composition tested. The particle charge was calculated as sum of the concentrations of the charged components (taken with the corresponding sign of (−) for TRP and PEGylated lipid, and (+) for cationic lipid) in the particle. It was assumed that both PEGylated lipid and cationic lipid are 100%/associated with particles, while TRP content in nanoparticles was calculated as TRPtotal (1-TRPfree %/100%). The data in FIG. 6B shows that the more positively charged particles retain treprostinil to a greater extent. Specifically, almost 100% retention (1-2% free TRP) is achieved when the particle charge becomes net positive.

FIG. 6C shows the amount of free treprostinil as a function of cationic lipid and total charge of the particles in the composition. The compositions tested in this experiment are provided in Table 7, and each included diC14dMA as the cationic lipid. Consistent with FIG. 6B, the amount of associated treprostinil correlates with increasing cationic lipid content. Stated another way, the amount of free treprostinil decreases with increasing cationic lipid concentration. Moreover, the amount of associated treprostinil is positively correlated with increasing positive particle charge.

TABLE 7

| | Cationic | (CL)/TRP | | CL/TRP mo- |
|---|---|---|---|---|
| | Lipid | molar ratio | | lar ratio in |
| Composition | (CL) | total | PEG-lipid | nanoparticle |
| TR0410A | diC14dMA | 0.71 | DSPE-PEG3K | 1.10 |
| TR0410B | diC14dMA | 0.97 | DSPE-PEG3K | 1.18 |
| TR0410C | diC14dMA | 1.20 | DSPE-PEG3K | 1.26 |
| TR0410D | diC14dMA | 1.46 | DSPE-PEG3K | 1.48 |
| TR0410E | diC14dMA | 1.70 | DSPE-PEG3K | 1.71 |
| TR0410F | diC14dMA | 1.91 | DSPE-PEG3K | 1.92 |

diC14dMA = ditetradecyldimethyl-ammonium bromide.
DSPE-PEG3K = distearoylphosphatidylethanolamine-PEG300.
CL/TRP mole ratio in nanoparticle was calculated by subtracting the measured TRP free and was used to determine the particle charge.

### Dialysis Assay

Table 8 provides the compositions used in the dialysis study. The results of this study can be used as an indication of which compositions might provide a sustained release profile in vivo.

TABLE 8
Compositions used in dialysis study.

| | | Hydrophobic | | Cationic |
|---|---|---|---|---|
| | TRP | additive | PEG-lipid | compound |
| Composition | (mol %) | (mol %) | (mol %) | (mol %) |
| T416 | 15% | Squalane | DSG-PEG2K | — |
| | | (20%) | (65%) | |
| T420 | 15% | Squalane | DSG-PEG2K | diC18dMA |
| | | (35%) | (20%) | (30%) |
| T426 | 18.3% | Squalane | DSG-PEG2K | diC18dMA |
| | | (25%) | (20%) | (36.7%) |
| T427 | 10% | Squalane | DSG-PEG2K | diC18dMA |
| | | (50%) | (20%) | (20%) |
| T428 | 3.3% | Squalane | DSG-PEG2K | diC18dMA |
| | | (70%) | (20%) | (6.7%) |
| T429 | 17.3% | Squalane | DSG-PEG2K | diC18dMA |
| | | (25%) | (23.1%) | (34.6%) |
| T430 | 11.5% | Squalane | DSG-PEG2K | diC18dMA |
| | | (50%) | (15.4%) | (23.1%) |
| T431 | 6.9% | Squalane | DSG-PEG2K | diC18dMA |
| | | (70%) | (9.3%) | (13.8%) |

TRP = treprostinil;
DSG-PEG2K = disteraroylglycerol-PEG2000;
diC18dMA = dioctadecyldimethyl ammonium bromide.

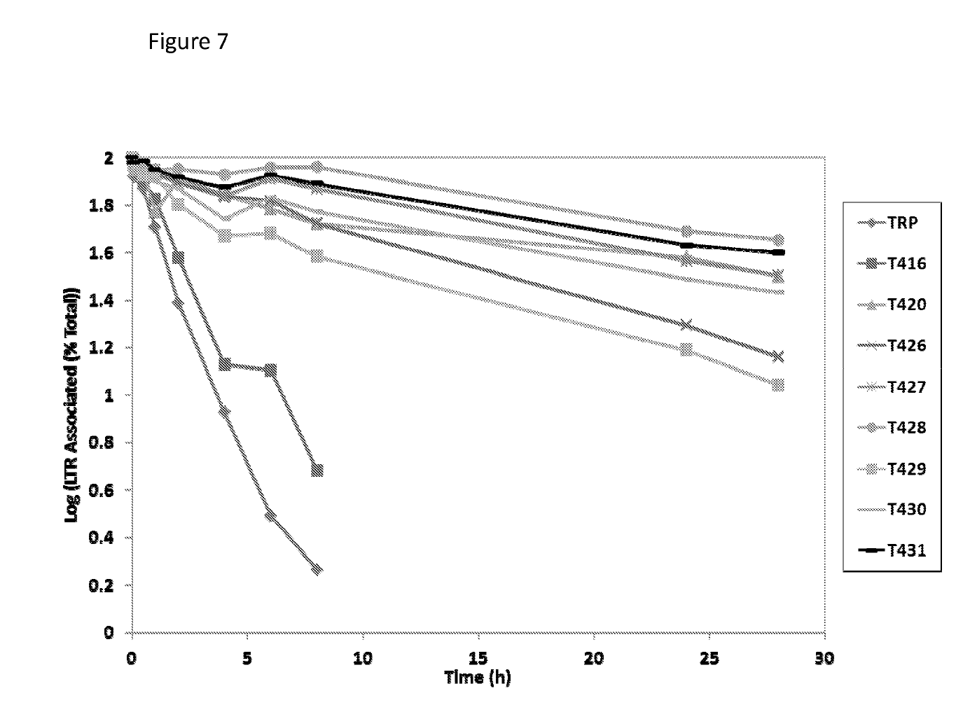

5 mL of each sample was dialyzed against 1 L of 1×PBS. 50 L sample was collected and tested by HPLC at different time points (FIG. 7). 250 μL sample was collected at 24 h. Each time a sample was collected, an equal volume of 1×PBS was added to the dialysis bag. Absorbance data was normalized to the calculated dilution factor. The dialysis membrane used had a 50 kDa MW cutoff.

Results of the study are provided at FIG. 7. Free treprostinil dialyzed out the fastest, as its kinetics was limited only by free diffusion through the dialysis membrane pores. Composition T416 (containing no cationic lipid) was released almost as fast confirming our previous observation that net positive charge is required for efficient retention of treprostinil. In contrast, T426, T427, T428, T429, T430 and T431 each dialyzed slowly, indicating that these compositions might be useful as sustained release compositions in vivo. The top three compositions were T428>T431>427, with treprostinil content 3.3%, 6.9%, and 10%, respectively. This suggests that with the TRP/cationic lipid ratio constant (2:1), compositions with lower TRP mol % retain treprostinil better and release it slower during dialysis.

### Example 4

### Measurement of Cyclic Adenosine Monophosphate (cAMP) Levels in CHO-K1 Cells in Response to Treprostinil Compositions

A cell based Chinese hamster ovary-K1 (CHO-K1) assay based on the GloSensor™ cAMP assay (Promega) was used to characterize the effect of treprostinil alkyl ester compounds on cAMP levels.

cAMP is a second messenger involved in signal transduction of G-protein coupled receptors (GPCRs) acting through Gα-s and Gα-i proteins. Because the treprostinil receptor is a GPCR, the assay provides an indication of whether the respective prostacyclin composition binds its receptor and activates the GPCR cell signaling cascade.

The GloSensor™ assay harnesses a genetically modified form of firefly luciferase into which a cAMP-binding protein moiety has been inserted. Upon binding of cAMP, a conformational change is induced leading to increased light output.

The EP2 prostanoid receptor was co-transfected with the GloSensor™ plasmid (Promega) into CHO-K1 cells as follows. CHO-K1 cells were harvested when the monoloayer was at 50-90% confluence. First, cells were washed with 5 mL PBS. Two mL of pre-warmed (37° C.) 0.05% trypsin-EDTA (Life Technologies, Cat #: 25300054) was added, and cells were dislodged by tapping the flask on the side. Next, 10 mL of antibiotic free growth media (Life Tech, Cat #: 31765092) containing 10% fetal bovine serum (FBS; Hyclone, Cat #: SH30071.03) was added, and cells were centrifuged at 250×g for 5 minutes at room temperature. The media was aspirated, and the cell pellet was resuspended in 10 mL of growth media. Cell number was determined using a hemacytometer. Each well of a culture treated 96 well flat bottom plate (Costar, Cat #: 3917) was seeded with 1×10⁴ cells per 100 μL antibiotic-free growth media. The cells were incubated overnight at 37° C. and 5% CO₂ in a water-jacketed incubator.

For small scale transfections of up to 20 wells, the pGLoSensor-22F cAMP plasmid (Promega, Cat #: E2301) (2 μg): (EP2) (10 ng) (Origene, Cat #: SC126558): pGEM-3Zf (+) (10 ng) (Promega, Cat #: P2271) ratio was diluted to a final concentration of 12.6 ng/μL (total plasmid) in Opti-MEM 1 reduced-serum medium (Life Technologies, Cat #: 1985062). Next, 6 μL of FuGENE HD transfection reagent (Promega, Cat #: E2311) was added to 160 L of diluted plasmid and mixed carefully by gentle pipetting. The complex was incubated at room temperature for 0 to 10 minutes, and then 8 μL of the complex was added per well of a 96 well white assay plate (Costar, Cat #: 3917) and gently mixed without disturbing the cell monolayer. The plates were incubated for 20-24 hours at 37° C. and 5% CO₂ in a water-jacketed incubator.

Following incubation, cells were treated and analyzed. For larger scale transfections, the aforementioned steps were scaled up accordingly, and cells were frozen following the last incubation. In order to prepare frozen transfected CHO-K1 cells, the media was aspirated from culture flasks and cells were rinsed with 5 mL PBS. As above, 2 mL of pre-warmed (37° C.) 0.05% trypsin-EDTA (Life Technologies, Cat #: 25300054) was added, and cells were dislodged by tapping the flask on the side. Next, 10 mL of antibiotic free growth media (Life Technologies, Cat #: 31765092) containing 10% FBS (Hyclone, Cat #: SH30071.03) was added, and cells were centrifuged at 250×g for 5 minutes at room temperature. Cell number was determined using a hemacytometer. The media was aspirated, and the cell pellet was resuspended in freezing media (Millipore, cat #: S-002-5F) at 2.5×10⁶ cells/vial. Transfected cells were incubated overnight at −80° C. before transfer to liquid nitrogen for long term storage. The frozen stocks were then thawed one day prior to use for assays, and cells were seeded at 2.5×10⁴ cells per well in 100 L of antibiotic-free complete media (F12 (Life Technologies, Cat #: 31765092)+10% FBS (Hyclone, Cat #: SH30071.03)). Following an overnight incubation at 37° C. and 5% CO₂ in a water-jacketed incubator, the cells were ready for use in cAMP response assays.

In preparation for cAMP measurement, the cells were equilibrated with the GloSensor cAMP reagent prior to treatment. For equilibration, the medium was carefully removed from the individual well. Next, 100 μL of equilibration medium (6% v/v of Glosensor Reagent stock solution (Promega, Cat #: E291), 10% FBS (Hyclone, Cat #: SH30071.03) and 88% CO₂ independent medium (Life Technologies, Cat #: 18045088)) was added per well of the 96-well plate, and added to the side of each well. The plate was then incubated for 2 hours at room temperature. A first pre-read measurement was taken using a microplate reader (MicroLumat Plus). Plates were incubated for an additional 10 minutes at room temperature, followed by a second pre-read measurement.

Working solutions of free treprostinil and treprostinil compositions were prepared at 10× concentration so that the final concentration was 1× once added to the cells. Following treatment, each plate was read every 5 minutes for the duration of the assay using a microplate reader (MicroLumat Plus). In order to determine the fold change in cAMP relative to the control, the transfection efficiency was first determined by dividing the second pre-read measurement by the average of the corresponding pre-read measurements. Next, the normalized relative light units (RLUs) of the samples were determined by dividing the plate read measurement by the transfection efficiency. The fold change in cAMP relative to the control was then determined by dividing the normalized RLU of the samples by the normalized RLU of the control.

Validation of cAMP Assay Using Free Treprostinil

The cAMP assay was validated using free treprostinil. Treprostinil (10 λM, 1 μM, 0.1 μM, 0.01 μM, 0.001 μM, 0.0001 μM, 0.00001 μM, and 0.000001 μM) was added to equilibrated CHO-K1 cells, and the cells were then incubated for 30 minutes. Luminescence was then measured at room temperature.

### Treprostinil Compositions

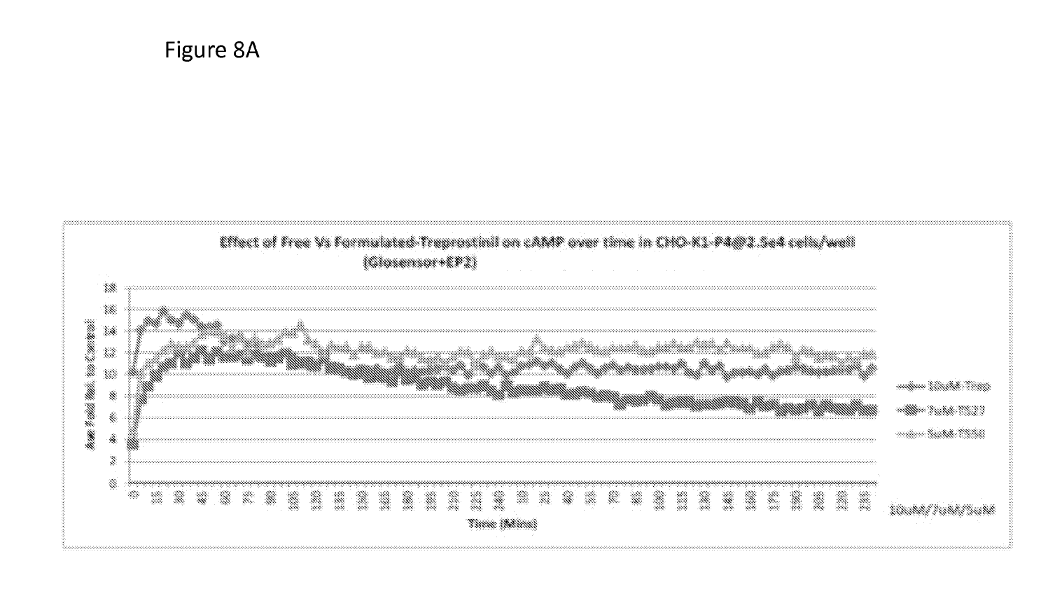

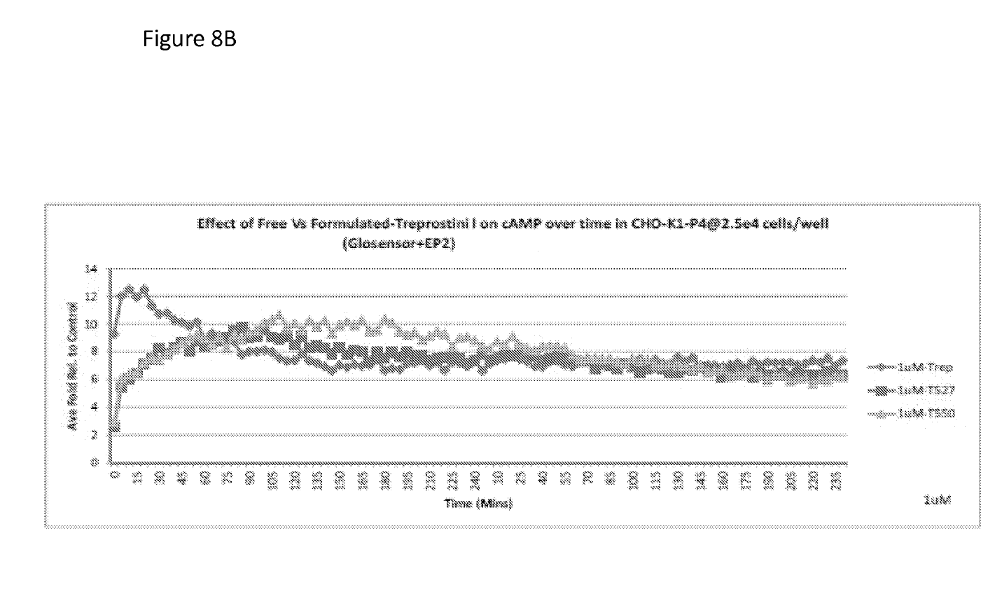

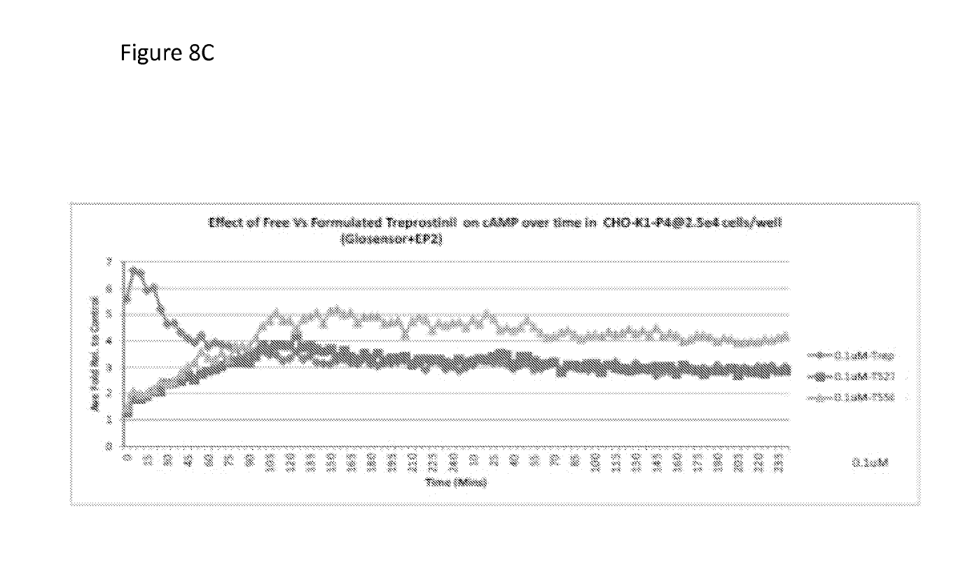

CHO-K1 cells co-transfected with the EP2 receptor and GloSensor™ plasmid were challenged with free treprostinil (10 μM) and treprostinil compositions T527 and T550 (Table 9) at the indicated concentrations. cAMP levels were then measured every 5 minutes over a time course of 8 hours as shown in FIGS. 8A-C.

TABLE 9
Treprostinil compositions used in GloSensor assays.

| | TRP | Hydrophobic | | Cationic | |
|---|---|---|---|---|---|
| | (mol | additive | PEG-lipid | compound | DOPC |
| Composition | %) | (mol %) | (mol %) | (mol %) | (mol %) |
| T527 | 5% | Squalane | Chol-PEG2K | diC18dMA | 10% |
| | | 60% | 10% | 15% | |
| T550 | 2% | Squalane | Chol-PEG2K | diC18dMA | 10% |
| | | 68% | 10% | 10% | |

Chol-PEG2K = Cholesterol-PEG2000.
diC18dMA = dioctadecyldimethyl ammonium bromide.

cAMP levels in response to the treprostinil compositions (2 μM) were equivalent to free treprostinil and the levels were sustained for at least 6 hours. The sustained cAMP level was not exhibited in response to free treprostinil.

### Nebulized Treprostinil Compositions

The cell based (CHO-K1) cAMP assay described above was also used to characterize the effect of nebulization of various treprostinil compositions on cAMP levels.

Nebulizer Aeroneb Pro (Aerogen) was used to nebulize treprostinil compositions. Desired volume of the formulation (usually 3 mL) was loaded to the mesh head of the nebulizer. The head was connected directly to the glass impinger with air-tight seal. Nebulization was carried out using factory settings until the entire sample was nebulized. After nebulization was complete, the head was disconnected; impinger capped and centrifuged 5 min at 600×g to settle the aerosol inside the impinger. The procedure provided nearly 100% yield in collecting the nebulized sample.

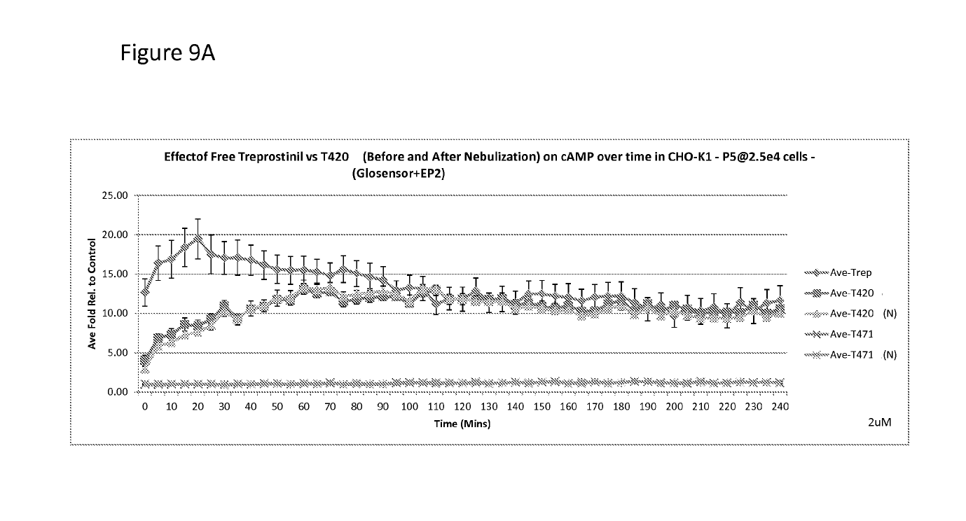

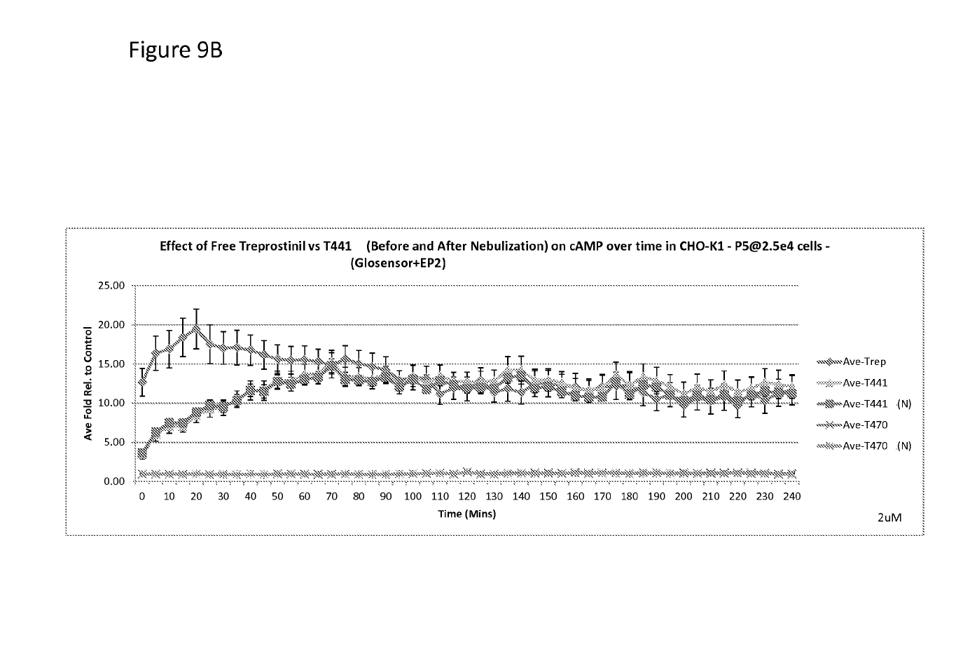

The compositions tested in this experiment are provided in Table 10, below, results in FIG. 9A-B. cAMP levels were measured every 5 minutes over a time course of 240 minutes.

TABLE 10
Treprostinil compositions used in the nebulization study.

| | | Hydrophobic | | Cationic |
|---|---|---|---|---|
| | TRP | additive | PEG-lipid | compound |
| Composition | (mol %) | (mol %) | (mol %) | (mol %) |
| T420 | 15% | Squalane | DSG-PEG2K | diC18dMA |
| | | 35% | 20% | 30% |
| T441 | 5% | Squalane | DSG-PEG2K | diC18dMA |
| | | 60% | 20% | 15% |
| T470 | — | Squalane | DSG-PEG2K | diC18dMA |
| | | 63% | 21% | 16% |
| T471 | — | Squalane | DSG-PEG2K | diC18dMA |
| | | 42% | 23% | 35% |

DSG-PEG2K = disteraroylglycerol-PEG2000.
diC18dMA = dioctadecyldimethyl ammonium bromide.

Results of these experiments using the 2 μM dose are provided at FIG. 8. cAMP response to the treprostinil composition T420 and T441 (2 μM) was greater than or equivalent to the response induced by free treprostinil (FIG. 9A, 9B). The cAMP levels in response to T420 and T441 compositions were sustained significantly longer than free treprostinil.

### Example 5

### Determination of the Effect of Treprostinil Composition on Cell Proliferation

In order to determine any effect of treprostinil compounds on cell proliferation, cell based assays using CHO-K cells and rat alveolar cells (NR8383 cells) were performed.

### CHO-K1 Cells

CHO-K1 cells were harvested when the cell monolayer was 50-90% confluent (use passage 4-11). Media was aspirated out of the flask, and cells were rinsed with 2 mL of F12 media. Next, 1 mL of pre-warmed (37° C.) 0.25% trypsin-EDTA (Life Technologies, Cat#: 25300054) was added, and cells were dislodged from the flask by tapping it on the side. Complete growth media (F12 (Life Technologies. Cat #: 31765092)+10% FBS (Hyclone, Cat #: SH30071.03)+1× Pen-Strep (Life Technologies, cat #15140-122) was then added at a volume of 10 mL. Cells were centrifuged at 250×g for 5 minutes at room temperature, and the media was aspirated. The cell pellet was resuspended in 10 mL complete growth media. Cell number was determined using a hemacytometer. Cells were then seeded at 2000 cells per well of a 96-well plate in 100 μL of complete growth media. The plate was incubated overnight at 37° C. and 5% CO₂ in a water-jacketed incubator.

The next day, 80 μL of fresh complete media was added to each well, and CHO-K1 cells were challenged with treprostinil compound and composition treatments. The working solutions were prepared at 10× concentration, and following 2 fold serial dilutions, 20 μL aliquots were added per well to arrive at a final 1X concentration. Following a 48 hour incubation at 37° C. and 5% CO₂ in a water-jacketed incubator, the inhibitory effect on cell proliferation was determined. Plates were analyzed using 20 μL of Presto Blue reagent (Life Technologies, cat #: A13262) per well. The reagent was mixed, and plates were incubated for 1 hour at 37° C. and 5% CO₂ in a water-jacketed incubator. Plates were read using either a CytoFluor Series 4000 (PerSeptive BioSystems) or Synergy Neo microplate reader (BioTek) with emission: 590 nm and excitation λ: 560 nm. The percent inhibition was determined using the following formula: % inhibition=100%−(treated samples/control×100%).

### NR8383 Cells

Rat alveolar NR8383 cells were harvested when the monolayer was 50-90% confluent (use passage 5-11). Because the NR8383 cells include both adherent and non-adherent cells, media was transferred to a 50 mL Falcon tube. To obtain the cells remaining in the flask, 2 mL of plain media was added, and the remaining cells were scraped out of the 75 cm² flask with a cell scraper and added to the 50 mL tube. Cells were centrifuged at 200×g for 5 minutes at room temperature, and the media was aspirated. The cell pellet was resuspended in 10 mL complete growth media (F12 (Life Technologies, Cat #: 31765092)+15% FBS—heat inactivated (Hyclone, Cat #: SH30071.03)+1× Pen-Strep (Life Technologies, cat #: 15410-122)). Cell number was determined using a hemacytometer. Cells were then seeded at 4000 cells per well of a 96-well plate in 100 μL of complete growth media. The plate was incubated overnight at 37° C. and 5% CO₂ in a water-jacketed incubator.

The next day, 80 μL of fresh complete media was added to each well, and the NR8383 cells were challenged with treprostinil compound treatments. Following a 72 hour incubation at 37° C. and 5% CO₂ in a water-jacketed incubator, the inhibitory effect on cell proliferation was determined. Measurements and calculations were made as described above for the CHO-K1 cells.

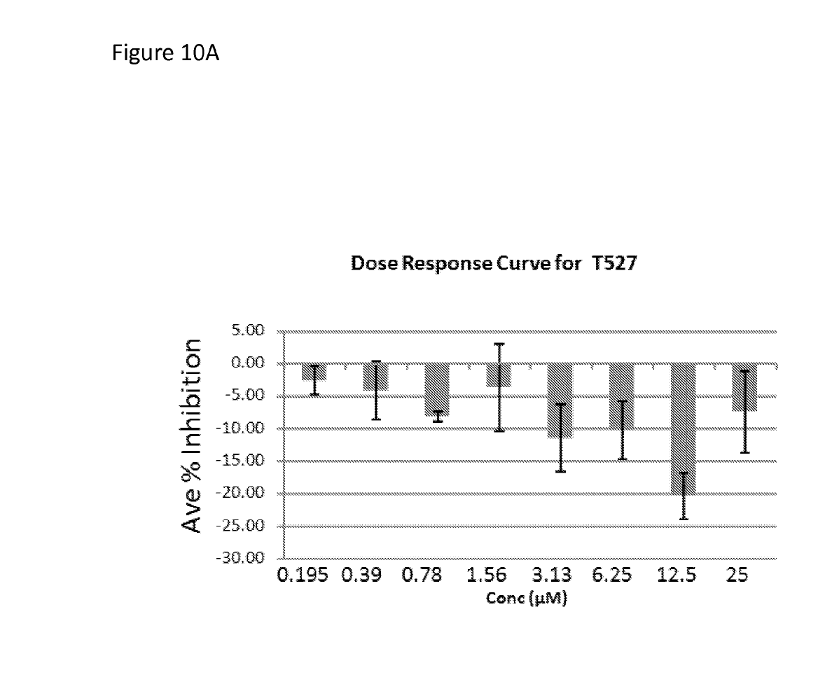

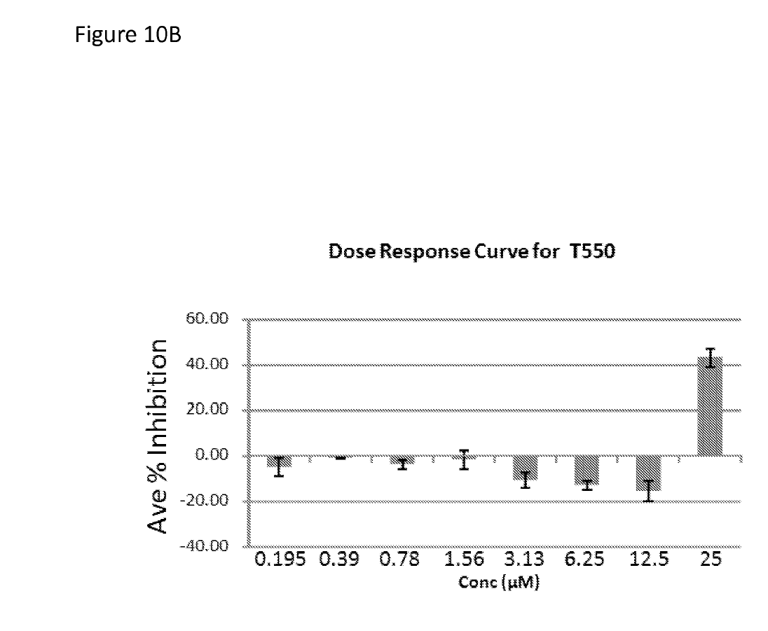

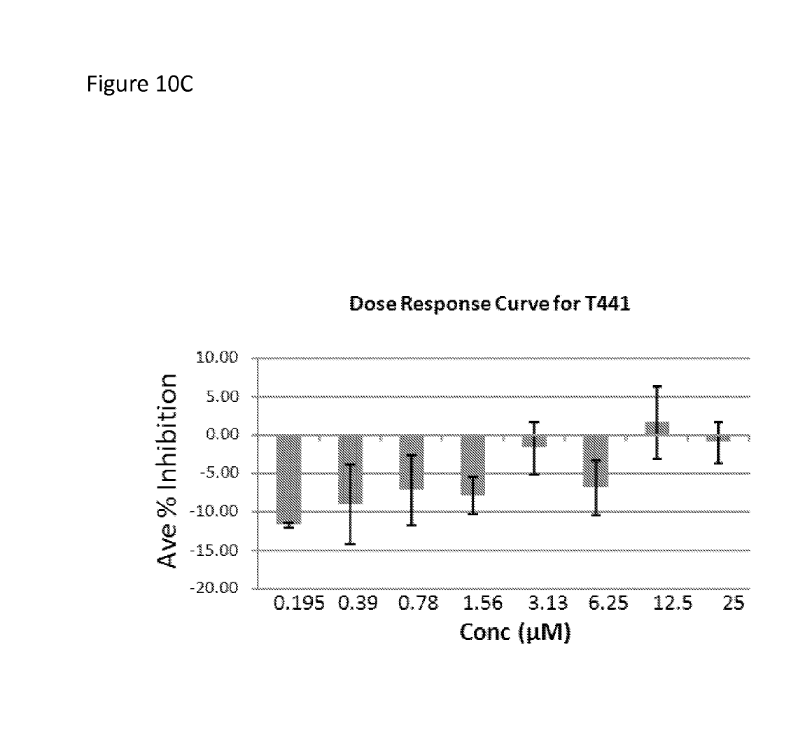

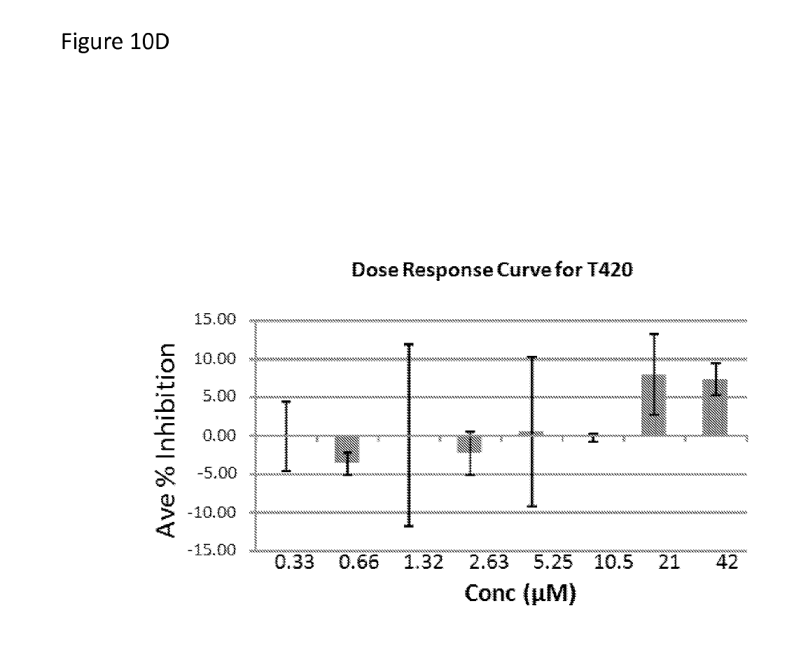

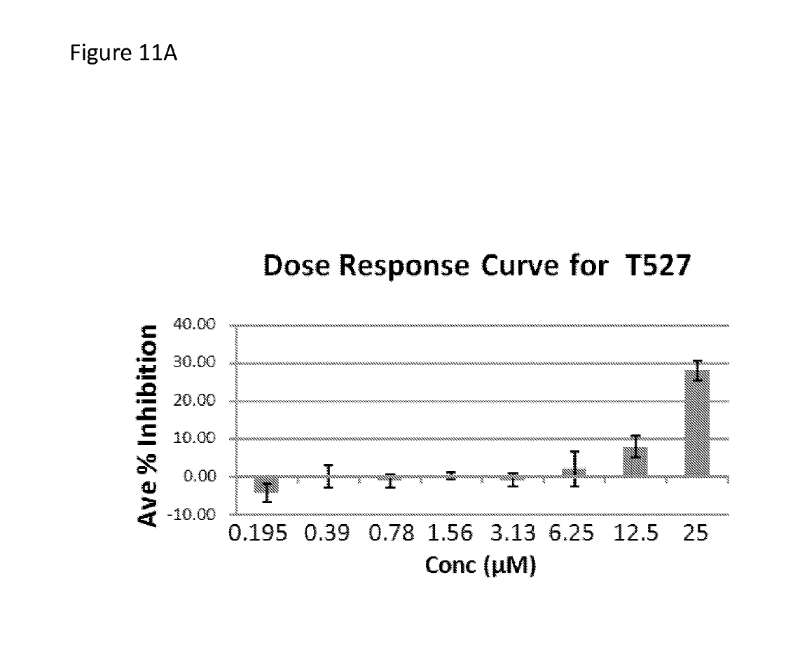

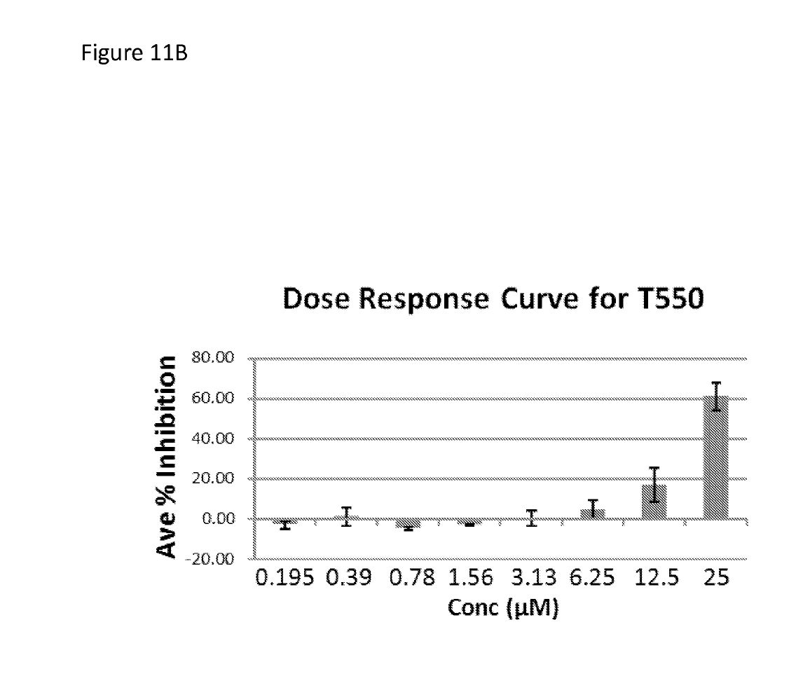

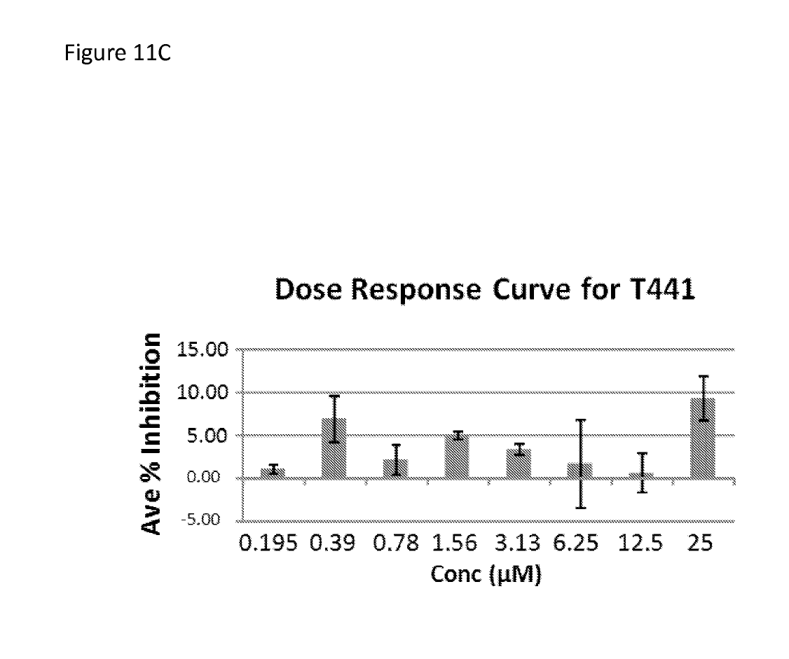

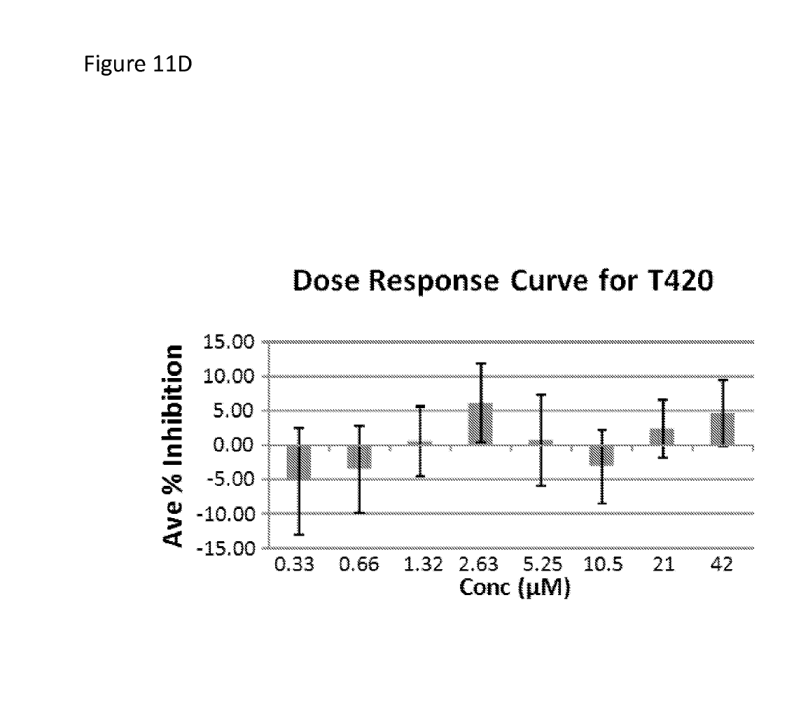

Four treprostinil compositions T441, T420, T550, and T527 were tested in the cell proliferation inhibition assays. (FIG. 10—CHO-K1, FIG. 11—NR8383). FIG. 10 shows the inhibitory effects of T527 (FIG. 10A), T550 (FIG. 10B), T441 (FIG. 10C) and T420 (FIG. 10D) on CHO-K1 cell proliferation. In particular, only T550 showed meaningful inhibitory effect on CHO-K1 cells (40%) at the highest concentration tested (25 μM). See FIG. 10A. Other compositions did not show a significant effect at all concentrations.

TABLE 11
Treprostinil compositions used in the cell proliferation assays.

| | | Hydrophobic | | Cationic |
|---|---|---|---|---|
| | TR | additive | PEG-lipid | compound |
| Composition | (mol %) | (mol %) | (mol %) | (mol %) |
| T420 | 15% | Squalane | DSG-PEG2K | diC18dMA |
| | | 35% | 20% | 30% |
| T441 | 5% | Squalane | DSG-PEG2K | diC18dMA |
| | | 60% | 20% | 15% |
| T550 | 5% | Squalane | Chol-PEG2K | diC18dMA |
| | | 60% | 10% | 15% |
| T527 | 2% | Squalane | Chol-PEG2K | diC18dMA |
| | | 68% | 10% | 10% |

DSG-PEG2K = disteraroylglycerol-PEG2000.
Chol-PEG2K = cholesterol-PEG2000.
diC18dMA = dioctadecyldimethyl ammonium bromide.

FIG. 11 summarizes the effect of the tested treprostinil compositions T527 (FIG. 11A), T550 (FIG. 11B), T441 (FIG. 11C) and T420 (FIG. 11D) on NR8383 cell proliferation. All tested treprostinil compositions showed some inhibition of cell proliferation from medium to the highest concentration. Specifically, of the four compositions, both T527 and T550 showed the significant inhibitory effect on NR8383 alveolar cell proliferation at 25 μM concentration, 30% and 60% correspondingly (FIGS. 11A and 11B).

### Example 6

### Treprostinil Composition In Vivo

The effect of treprostinil compositions in vivo was determined by using rat models. Young male rats Sprague Dawley (Charles River) were used for the study. Rats anesthetized with ketamine/xylazine, placed on a heating pad and after surgical isolation and catheterization of the trachea, mechanically ventilated throughout the study.

A catheter was placed in the femoral artery for measurement of systolic (sys) and diastolic (dias) blood pressures. A thoracotomy was performed and a catheter inserted into the right ventricle and positioned in the pulmonary artery for the measurement of pulmonary arterial systolic and diastolic blood pressures. Oxygen saturation (SaO2) was measured with a pulse oximeter placed on the paw.

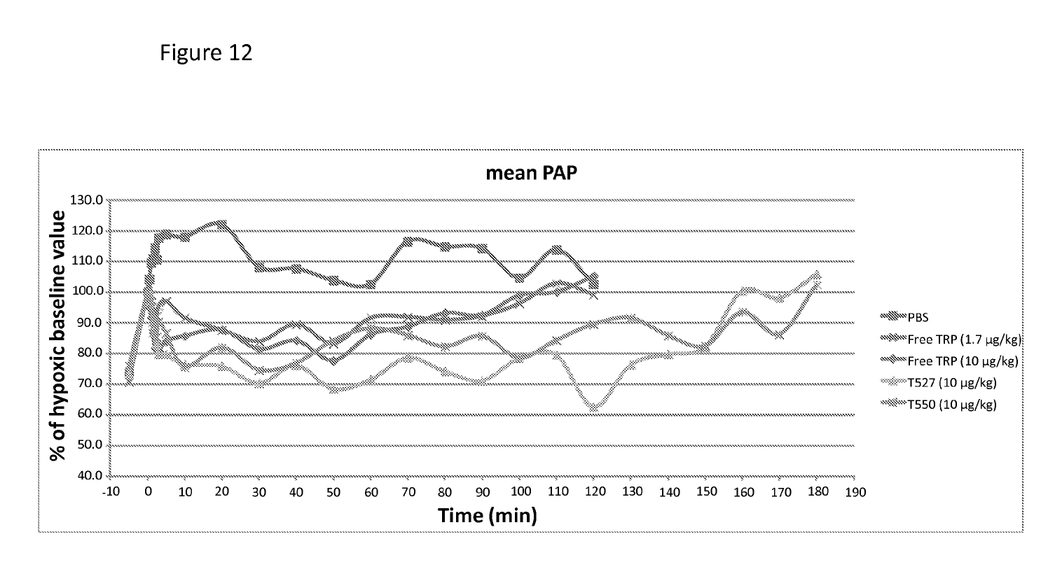

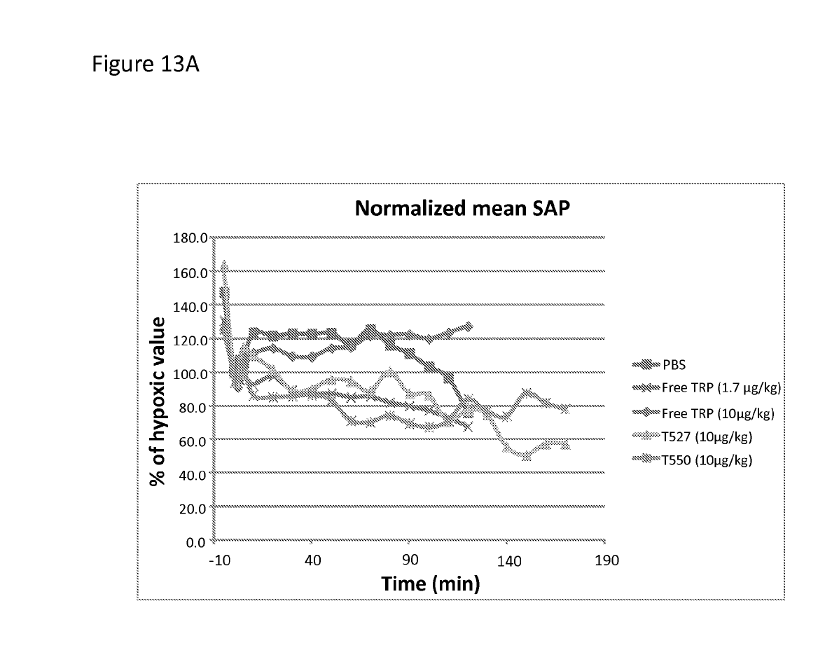

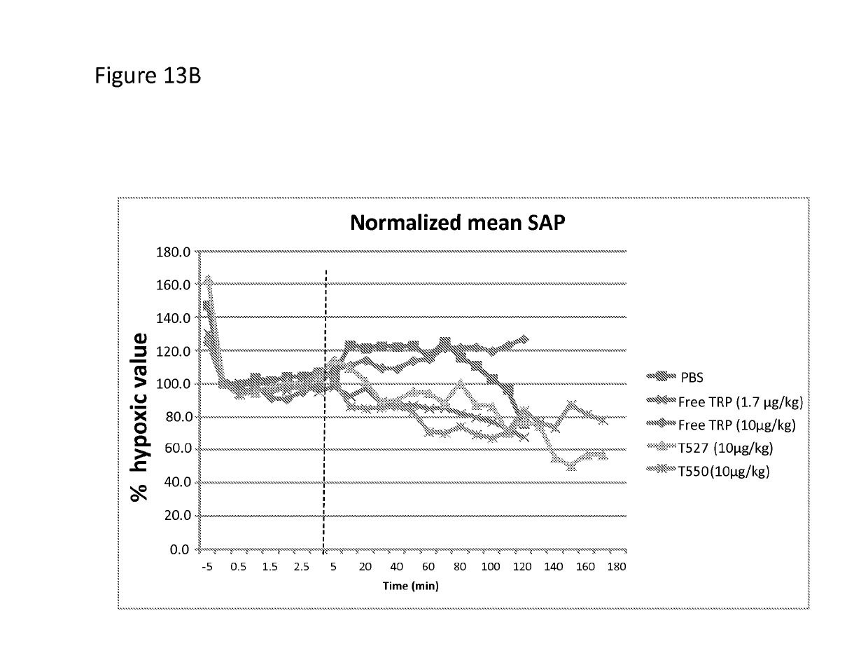

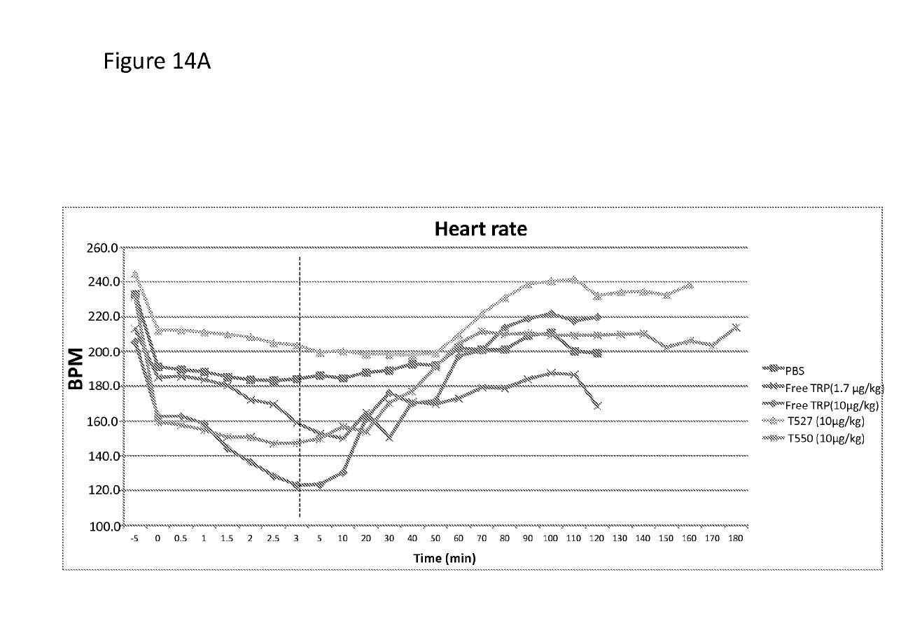

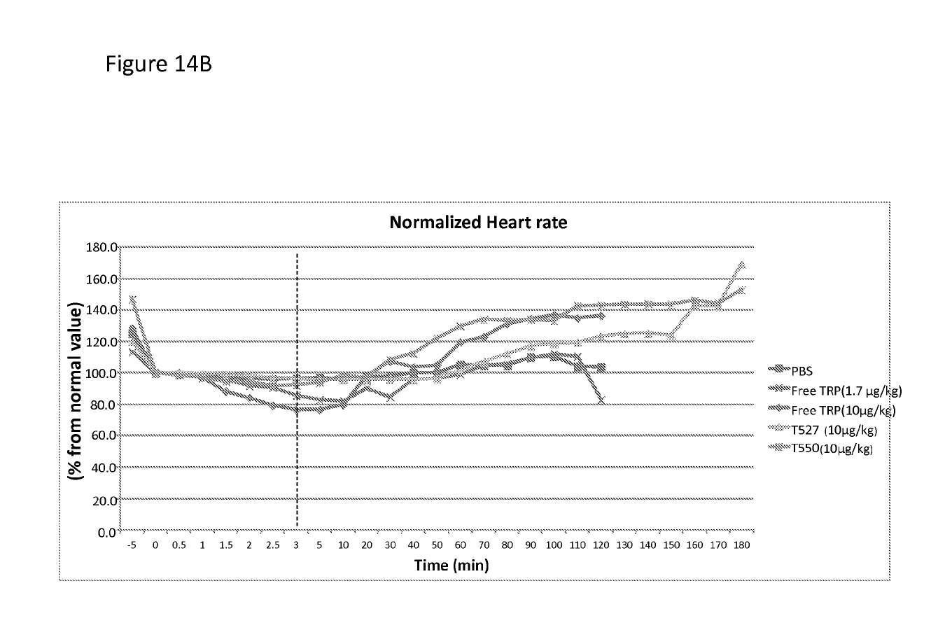

With the rats ventilated on room air (FIO2=0.21), cardiovascular measurements were made under these normoxic conditions. In order to induce hypoxia the FIO2 was reduced over a 30 min. period until SaO2 fell to values between 50-60%, and a baseline hypoxia value for each of the parameters was determined. Groups of four rats each received either PBS, free treprostinil (1.7 μg/kg and 10 μg/kg). T527 (10 μg/Kg) or T550 (10 μg/kg). The target dose varied slightly by weight due to the differences in molecular weight of the treprostinil derivative compositions as shown in FIGS. 12-14. The actual achieved lung dose was about 5× lower than provided in FIG. 12 (e.g., administration of 10 μg/kg yielded about 2 μg/kg in the lungs). The normalized variation of mean PAP (mPAP) is shown as a percentage from the hypoxic baseline value at (T=0) in FIG. 12. The hypoxic baseline PAP value was 100%, and the changes in pressure were measured in comparison to the hypoxic baseline. The normalized variation of mean SAP (mSAP) is shown as a percentage from the hypoxic baseline value in FIG. 13A-B. Heart rate is shown in FIG. 14A-B as a percentage of the hypoxic baseline value over time.

The various treatments were delivered (via inhalation of nebulized drug to the lungs of the rats. The pulmonary arterial pressure (PAP), systemic arterial pressure (SAP), and heart rate of the rats were measured continuously for 180 minutes. The PAP signal was collected at 200 points per second.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1.-118. (canceled)

119. A pharmaceutical composition comprising a prostacyclin analog, a cationic compound and a surfactant.

120. The pharmaceutical composition of claim 119, wherein the prostacyclin analog is epoprostenol, treprostinil or iloprost.

121. The pharmaceutical composition of claim 119, wherein the prostacyclin analog is treprostinil.

122. The pharmaceutical composition of claim 119, wherein the pharmaceutical composition comprises a plurality of particles comprising the prostacyclin analog and the cationic compound.

123. The pharmaceutical composition of claim 119, wherein the cationic compound is alkyl-ammonium, alkylpolyammonium, linear polyamine, linear polyethylenimine, branched polyethylenimine, poly-L-lysine, trimethyl-poly-glucosamine, a multivalent metal ion, N,N′-dihexadecyl-1,2-ethanediamine, tetraethylhexadecane-1,16-diamine, hexadecane-1,16-bis(trimethylammonium bromide), dimethyldioctadecylammonium bromide (DDAB), dimethyldihexadecylammonium chloride, N-[1-(2,3-dioleoyloxy)propyl]-N,N,N-trimethylammonium methyl sulfate (DOTAP), N-[1-(2,3-dioleyloxyl)propyl]-N,N,N-trimethylammonium chloride (DOTMA), 1,2-distearoyl-3-(trimethylammonio)propane chloride (DSTAP), dimyristoyltrimethylammonium propane (DMTAP), or dioctadecyldimethylammonium bromide (DODAB).

124. The pharmaceutical composition of claim 119, wherein the cationic compound is a cationic lipid.

125. The pharmaceutical composition of claim 119, wherein the surfactant is a polyoxyethyleneglyocol-lipid.

126. The pharmaceutical composition of claim 119, wherein the surfactant is an anionic surfactant.

127. The pharmaceutical composition of claim 122, wherein the plurality of particles further comprise the surfactant.

128. The composition of claim 122, wherein the plurality of particles is a plurality of solid lipid particles.

129. The composition of claim 128, wherein the mean diameter of the plurality of solid lipid nanoparticles is about 1 nm to about 1000 nm.

130. A method for treating pulmonary hypertension in a patient in need thereof, comprising administering to the patient an effective amount of a pharmaceutical composition comprising a prostacyclin analog, a cationic compound and a surfactant.

131. The method of claim 130, wherein the pulmonary hypertension is pulmonary arterial hypertension.

132. The method of claim 130, wherein the pulmonary hypertension is group II pulmonary hypertension.

133. The method of claim 130, wherein the pulmonary hypertension is group III pulmonary hypertension.

134. The method of claim 130, wherein the pulmonary hypertension is group IV pulmonary hypertension.

135. The method of claim 130, wherein the pulmonary hypertension is group V pulmonary hypertension.

136. The method of claim 130, wherein the prostacyclin analog is treprostinil.

137. The method of claim 130, wherein administration is oral, parenteral, subcutaneous, inhalation, intravenous, or infusion administration.

138. The method of claim 130, wherein the prostacyclin analog is epoprostenol, treprostinil or iloprost.

139. The method of claim 130, wherein the prostacyclin analog is treprostinil.

140. The method of claim 130, wherein the pharmaceutical composition comprises a plurality of particles comprising the prostacyclin analog and the cationic compound.

141. The method of claim 130, wherein the cationic compound is alkyl-ammonium, alkylpolyammonium, linear polyamine, linear polyethylenimine, branched polyethylenimine, poly-L-lysine, trimethyl-poly-glucosamine, a multivalent metal ion, N,N′-dihexadecyl-1,2-ethanediamine, tetraethylhexadecane-1,16-diamine, hexadecane-1,16-bis(trimethylammonium bromide), dimethyldioctadecylammonium bromide (DDAB), dimethyldihexadecylammonium chloride, N-[1-(2,3-dioleoyloxy)propyl]-N,N,N-trimethylammonium methyl sulfate (DOTAP), N-[1-(2,3-dioleyloxyl)propyl]-N,N,N-trimethylammonium chloride (DOTMA), 1,2-distearoyl-3-(trimethylammonio)propane chloride (DSTAP), dimyristoyltrimethylammonium propane (DMTAP), or dioctadecyldimethylammonium bromide (DODAB).

142. The method of claim 130, wherein the cationic compound is a cationic lipid.

143. The method of claim 130, wherein the surfactant is a polyoxyethyleneglyocol-lipid.

144. The method of claim 130, wherein the surfactant is an anionic surfactant.

145. The method of claim 140, wherein the plurality of particles further comprise the surfactant.

146. The method of claim 140, wherein the plurality of particles is a plurality of solid lipid particles.

147. The method of claim 146, wherein the mean diameter of the plurality of solid lipid nanoparticles is about 1 nm to about 1000 nm.
